SEQUENCE LISTING (29 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 38 aa, Homo sapiens
MDVFMKGLSKAKEGVVAAAEKTKQGVAEAAGKTKEGVL

SEQ ID NO: 3 - protein, 38 aa, Homo sapiens
MDVFMKGLSMAKEGVVAAAEKTKQGVTEAAEKTKEGVL

SEQ ID NO: 4 - protein, 38 aa, Homo sapiens
MDVFKKGFSIAKEGVVGAVEKTKQGVTEAAEKTKEGVM

SEQ ID NO: 5 - protein, 38 aa, synthetic construct
MDVFEEGLSKAEEGVVAAAEKTKQGVAEAAGKTKEGVL

SEQ ID NO: 6 - protein, 38 aa, synthetic construct
MDVFKKGFSIADEGVVGAVEKTDQGVTEAAEKTKEGVM

SEQ ID NO: 7 - protein, 38 aa, synthetic construct
MDVFKAGFSIAAEGVVGAVEATAQGVTEAAEATAEGVM

SEQ ID NO: 8 - protein, 38 aa, synthetic construct
MDVFKKGFSIAKEGVVGAVEKTKQGVTEMAEKTKEGVM

SEQ ID NO: 9 - protein, 38 aa, synthetic construct
MDVFKKGFSAAKEGVVGAVEKTKQGVTEAAEKTKEGVM

SEQ ID NO: 10 - protein, 38 aa, synthetic construct
MDVFKKGFSIPKEGVVGAVEKTKPGVTEAAEKTKEGVM

SEQ ID NO: 11 - protein, 38 aa, synthetic construct
MDVFKKGFSIAKEGVVGAVEKTKQGVTEAAKKTKEGVM

SEQ ID NO: 12 - protein, 38 aa, synthetic construct
MGVFKKGFSIADEGVVGAVEKTDQGVTEAAEKTKEGVM

SEQ ID NO: 13 - protein, 38 aa, synthetic construct
MPVFKKGFSIADEGVVGAVEKTDQGVTEAAEKTKEGVM

SEQ ID NO: 14 - protein, 20 aa, synthetic construct
MDVFKKGFSIADEGVVGAVE

SEQ ID NO: 15 - protein, 25 aa, synthetic construct
MDVFKKGFSIADEGVVGAVEKTDQG

SEQ ID NO: 16 - protein, 27 aa, synthetic construct
MDVFKKGFSIADEGVVGAVEKTDQGVT

SEQ ID NO: 17 - protein, 18 aa, synthetic construct
MDVFKKGFSIADEGVVGA

SEQ ID NO: 18 - protein, 15 aa, synthetic construct
MDVFKKGFSIADEGV

SEQ ID NO: 19 - protein, 11 aa, synthetic construct
MDVFKKGFSIA

SEQ ID NO: 20 - protein, 20 aa, synthetic construct
MDVYKKGYSIADEGVVGAVE

SEQ ID NO: 21 - protein, 20 aa, synthetic construct
MDVAKKGASIADEGVVGAVE

SEQ ID NO: 22 - protein, 20 aa, synthetic construct
MPVFKKGFSIADEGVVGAVE

SEQ ID NO: 23 - protein, 20 aa, synthetic construct
MDVFKDGFSIADEGVVGAVE

SEQ ID NO: 24 - protein, 20 aa, synthetic construct
MPVFDDGFSIADEGVVGAVE

SEQ ID NO: 25 - protein, 10 aa, synthetic construct
MDVFKKGFSI

SEQ ID NO: 26 - protein, 9 aa, synthetic construct
MDVFKKGFS

SEQ ID NO: 27 - protein, 20 aa, synthetic construct
MDVFKKGFDIADEGVVGAVE

SEQ ID NO: 28 - protein, 38 aa, synthetic construct
MDVFMKGLDKAKEGVVAAAEKTKQGVAEAAGKTKEGVL

SEQ ID NO: 29 - protein, 31 aa, synthetic construct
RMKQLEDKVEELLSKNYHLENEVARLKKLVG

(19) United States
(12) Patent Application Publication
(10) Pub. No.: US 2025/0059578 A1
(43) Pub. Date: Feb. 20, 2025

(54) SEQUENCES AND METHODS FOR PRODUCTION OF RECOMBINANT BIOLOGICAL MOLECULES IN VESICLES

(71) Applicants: University of Kent, Kent (GB); Fujifilm Diosynth Biotechnologies UK Limited, Billingham (GB)

(72) Inventors: Daniel MULVIHILL, Kent (GB); Karen BAKER, Kent (GB); Tara EASTWOOD, Kent (GB); Christopher LENNON, Billingham (GB)

(21) Appl. No.: 18/719,423

(22) PCT Filed: Dec. 15, 2022
(86) PCT No.: PCT/GB2022/053239

§ 371 Date: Jun. 13, 2024

(30) Foreign Application Priority Data
Dec. 17, 2021 (GB) 2118435.3

(51) Int. Cl. C12P 21/00; C07K 14/47; C12N 15/62; C12N 15/70

(52) U.S. Cl. CPC C12P 21/00; C07K 14/4711; C12N 15/62; C12N 15/70; C07K 2319/50; C07K 2319/73

(57) ABSTRACT

Described is a method and component parts of a recombinant expression system for the expression of a biological molecule. In particular, an isolated vesicle nucleating polypeptide (VNp) comprising an amphipathic alpha helix polypeptide derived from the amino terminus of synuclein isoforms and variants thereof is used to promote, enhance or increase the formation of vesicles in the expression system to enable purification of the biological molecule.

The present invention relates to a technology to enhance the production of biological molecules in a cellular expression system. In particular, there is described a system to export recombinant proteins from engineered cells into induced extracellular vesicles.

E. coli is an attractive system for recombinant protein production in both academic and industrial scales (Jan, A. T. (2017) Front. Microbiol. 8, 7692-11; Gerritzen, M. J. H. et al (2017) Biotechnology Advances 35, 565-574). Not only is it cheap and easy to culture in batches to high densities, but a wide range of strains, reagents, promoters and tools have also been developed to facilitate the production of functional proteins in E. coli. The application of synthetic biology strategies is now overcoming limitations commonly associated with the application of post-translational modifications and correct folding of complex proteins (Kim, J. H. et al (2015) Semin. Cell Dev. Biol. 40, 97-104). As for all expression systems, there is always a need to improve protein yield and improve the efficiency of protein purification.

Gram-negative bacteria produce Outer Membrane Vesicles (OMV) to facilitate extracellular communication and interactions (e.g. quorum sensing). OMVs are small spherical lipid bilayers (˜100 nm diameter) released from the outer membrane. Detailed mass spectroscopic and chromatography analysis has defined the composition of natural OMVs which have been shown to contain DNA/RNAs, protein or other molecules from the cell wall, periplasm or cytosol regions of the cell (Jan, A. T. supra). Natural OMVs are enriched at their surface with proteins that enhance their abilities to fuse with host cells, making them attractive vessels for drug and vaccine delivery applications (Gerritzen, M. J. H. et al supra). OMVs are equivalent to exosomes or extracellular vesicles from mammalian or Gram-positive bacteria (Kim, J. H. et al supra). OMVs originate from a localised loss of interaction between the asymmetric lipid bilayer of the outer membrane (OM; Konovalova, A. et al (2017) Annu. Rev. Microbiol. 71, 539-556) and the peptidoglycan (PG) layer brought about by localised curvature of the outer membrane caused by changes in turgor pressure, temperature, or localised concentrated protein interactions (Jan, A. T. supra; Kim, J. H. et al supra). They are naturally produced at a low yield during bacterial growth cycle, with a slight increase during stationary phase. It is possible to direct packaging of proteins into natural OMVs. However, due to low OMV abundance, only relatively low protein yields have been observed to date (Alves, N. J. et al (2015) ACS Appl. Mater. Interfaces 7, 24963-24972). Large scale deletion and overexpression screens have revealed it is possible to change the composition of the outer membrane itself, thereby to improve yield of membrane proteins and modulate OMV production (McBroom. A. J. et al (2006) J. Bacteriol. 188, 5385-5392; Miroux, B. & Walker, J. E. (1996) Journal of Molecular Biology 260, 289-298; Baker, J. L. et al (2014) Curr. Opin. Biotechnol. 29, 76-84). Intriguingly, expression of caveolin-1 in E. coli cells leads to the accumulation of small circular vesicles (40-50 nm diameter) within the cytoplasm of each cell, likely to have formed from inward budding of the cell's inner membrane.

The ability to reprogram a cell to direct the packaging of specific molecules into discrete membrane envelopes is a major objective for synthetic biology. This controlled packaging into membrane vesicles will allow biologists to create a plethora of new technologies, which could be applied in both biotechnology and medical industries. These include the generation of novel metabolic factories within a cell for energy production; for rapidly packaging toxic proteins into contained environments before they have a chance to harm any normal metabolic activities, so they can be purified for use in subsequent pharmaceutical applications; the creation of protective packages filled with difficult to isolate biomolecules, which can be kept in a stable environment to allow their storage and purification; and also generate simple vehicles for delivery of drugs and vaccines to the patient.

It is against this background that the present invention has been devised.

Accordingly, the present invention encompasses a polypeptide sequence and method that provides a simple mechanism for targeted release of specific membrane packaged biological molecules such as proteins into cell media which can be continuously isolated from active cultures. This results in a significant increase in yields of functional soluble molecules, such as proteins, from cultures and facilitates efficient downstream processing for a wide range of biotechnology applications.

In particular, the present invention relates to an isolated vesicle nucleating polypeptide (VNp) comprising an amphipathic alpha helix polypeptide derived from the amino terminus of synuclein isoforms and variants thereof. Expressed in another way, the invention resides in variants of a short amphipathic alpha helix polypeptide that is based on the amino terminus of synuclein isoforms and designed variants thereof.

The synuclein family includes three known proteins: alpha-synuclein, beta-synuclein, and gamma-synuclein. All synucleins have a highly conserved alpha-helical lipid-binding motif in common with similarity to the class-A2 lipid-binding domains of the exchangeable apolipoproteins.

The full-length endogenous Hs alpha-synuclein has the following sequence (GenBank sequence: AAL15443.1):

(SEQ ID NO: 1)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

The VNp may have between 9 and 140 amino acids, such as from 15 to 140 amino acids, preferably between 16 and 40 amino acids, more preferably about 20 amino acids or about 38 amino acids.

For example, the amino terminal of Hs alpha-synuclein has the following sequence:

(SEQ ID NO: 2)
MDVFMKGLSKAKEGVVAAAEKTKQGVAEAAGKTKEGVL

The amino terminal of Hs beta-synuclein has the following sequence:

(SEQ ID NO: 3)
MDVFMKGLSMAKEGVVAAAEKTKQGVTEAAEKTKEGVL

The amino terminal of Hs gamma-synuclein has the following sequence:

(SEQ ID NO: 4)
MDVFKKGFSIAKEGVVGAVEKTKQGVTEAAEKTKEGVM

Variants of the amino-terminal sequence may include changing one or more basic lysine residues on the membrane interface to acidic or neutral residues. Such changes stabilise the interaction with the membrane. Examples of such sequences are as follows in which differences from the wild type protein sequence are bold and underlined:

(SEQ ID NO: 5)
MDVFEEGLSK AEEGVVAAAE KTKQGVAEAA GKTKEGVL
(SEQ ID NO: 6)
MDVFKKGFSI ADEGVVGAVE KTDQGVTEAA EKTKEGVM
(SEQ ID NO: 7)
MDVFKAGFSI AAEGVVGAVE ATAQGVTEAA EATAEGVM

While mutations and variations may be designed, sequence variants may include mutations and changes that are created through a random mutagenesis screen, for example by looking specifically for mutations that increase export of a particular protein or marker in culture media.

Examples of variants identified using this method include the following sequences:

(SEQ ID NO: 8)
MDVFKKGFSI AKEGVVGAVE KTKQGVTEMA EKTKEGVM
(SEQ ID NO: 9)
MDVFKKGFSA AKEGVVGAVE KTKQGVTEAA EKTKEGVM
(SEQ ID NO: 10)
MDVFKKGFSI PKEGVVGAVE KTKPGVTEAA EKTKEGVM
(SEQ ID NO: 11)
MDVFKKGFSI AKEGVVGAVE KTKQGVTEAA KKTKEGVM

Examples of other suitable variants take one of the variant sequences and add further variations, such as the following sequences:

(SEQ ID NO: 12)
MGVFKKGFSI ADEGVVGAVE KTDQGVTEAA EKTKEGVM
(SEQ ID NO: 13)
MPVFKKGFSI ADEGVVGAVE KTDQGVTEAA EKTKEGVM

Examples of suitable shorter variants include the following sequences:

(SEQ ID NO: 14)
MDVFKKGFSI ADEGVVGAVE
(SEQ ID NO: 15)
MDVFKKGFSI ADEGVVGAVE KTDQG
(SEQ ID NO: 16)
MDVFKKGFSI ADEGVVGAVE KTDQGVT
(SEQ ID NO: 17)
MDVFKKGFSI ADEGVVGA
(SEQ ID NO: 18)
MDVFKKGFSI ADEGV
(SEQ ID NO: 19)
MDVFKKGFSI A
(SEQ ID NO: 20)
MDVYKKGYSI ADEGVVGAVE
(SEQ ID NO: 21)
MDVAKKGASI ADEGVVGAVE
(SEQ ID NO: 22)
MPVFKKGFSI ADEGVVGAVE
(SEQ ID NO: 23)
MDVFKDGFSI ADEGVVGAVE
(SEQ ID NO: 24)
MPVFDDGFSI ADEGVVGAVE
(SEQ ID NO: 25)
MDVFKKGFSI
(SEQ ID NO: 26)
MDVFKKGFS
(SEQ ID NO: 27)
MDVFKKGFDI ADEGVVGAVE
(SEQ ID NO: 28)
MDVFMKGLDK AKEGVVAAAE KTKQGVAEAA GKTKEGVL

It will be understood that the invention encompasses any number of residues within the ranges stated. For example, the sequence may specifically have 20, 21, 24, 28, 33 or 38 amino acid residues.

Alternatively or in addition, the amino terminal sequence may include modifications and/or mutations to (a) change the stability of the helix terminus, and/or (b) prevent amino-terminal acetylation of the amino-terminus. The latter variation may be of particular benefit in eukaryote (especially mammalian) expression systems as it is often not possible to prevent amino-terminal acetylation in these cells without generating knock-out cell lines. In embodiments employing E. coli, if desired, amino-terminal acetylation of a target protein can be brought about using an inducible recombinant system developed in the Mulvihill lab as described in Eastwood T. A. et al (2017) FEBS Letters. 591, 833-44; Johnson M. et al (2010) PLOS One. 5, e15801, to induce this post-translational modification, which may beneficially impact the structure and function of many eukaryote proteins.

Alternatively or in addition, the sequence may include one or more synthetic amino acids.

Optionally, the polypeptide may be acetylated at its amino terminus. This feature promotes the rapid formation and internalisation of vesicle structures into the cytosol of a host cell, and thereby sequester toxic and/or insoluble proteins out of the cytosol.

The VNp may further comprise a leucine zipper sequence. Preferably, the leucine zipper sequence is at the carboxyl end of the polypeptide. The addition of a dimerising leucine zipper sequence can, in some embodiments, enhance export and/or expression for example, of otherwise difficult to express (e.g. toxic or insoluble) proteins. An example of a suitable leucine zipper sequence is as follows:

(SEQ ID NO: 29)
RMKQLEDKVE ELLSKNYHLE NEVARLKKLV G

The present invention also resides in a fusion protein comprising a vesicle nucleating polypeptide as described herein above fused to the amino terminus of a biological molecule, such as a recombinant protein. While a fusion protein having a vesicle nucleating polypeptide and a single biological molecule is exemplified herein, the present invention encompasses the export of multiple proteins via vesicles, for example in a tandem fusion (such as a fluorescent protein together with one or more target protein), or by export of complexes of separate VNp-proteins (as demonstrated by Bi-Molecular Fluorescence Complementation (BiFC fluorescence) in vesicles) or by leucine-zipper dimerised proteins. For the avoidance of doubt, BiFC fluorescence is obtained by i) fusing the first half of a fluorescent protein to VNp, ii) fusing the carboxyl half of the fluorescent protein to VNp, and iii) expressing the two fusions together. If the two halves of the fluorescent protein are brought together, they fluoresce. When fluorescence is seen in the vesicles, it can be concluded that the vesicles contain both individual proteins.

In many embodiments, the vesicle nucleating polypeptide may be fused directly to the biological molecule. The VNp interacts with a host cell membrane to promote formation of vesicular structures derived from the cellular outer membrane and containing the biological molecule fused to the VNp sequence. In certain preferred embodiments, the VNp may be fused to the biological molecule via a protease cleavage site. Protease cleavage sites include those for viral proteases, including Etch virus proteases, such as Tev (tobacco etch virus), viral 3C-like proteases, such as Human Rhinovirus HRV 3C; Enterokinase; Factor Xa and Thrombin. In many preferred embodiments, the protease cleavage site may be scarless, with all of the recognition site removed. Examples of such cleavage sites are those for signal peptidases such as E. coli LepB and, in particular, the lanthipeptidases disclosed in WO2015/175576, such as Bacillus licheniformis LicP. Eukaryote signal peptidases may also be employed, or further processing enzymes such as Pichia Kex2 or Ste13. Cleavage sites for membrane-bound, and preferably cytosolic, proteases may be employed. In many embodiments, the protease recognition site comprises a sequence of five or six amino acids. Such an arrangement enables in-vesicle proteolytic cleavage and processing of the biological molecule.

In some embodiments, an additional VNp comprising a leucine zipper sequence may be co-expressed with a fusion protein comprising a vesicle nucleating polypeptide comprising a leucine zipper sequence fused to the amino terminus of a biological molecule. The additional VNp may be expressed from the same vector as the vesicle nucleating polypeptide fused to the amino terminus of a biological molecule, or the additional VNp may be expressed from different vectors, for example plasmids or episome, or may be chromosomally expressed.

In further embodiments, a vesicle nucleating polypeptide comprising a leucine zipper sequence may be fused to the amino terminus of a protease selected to correspond with the protease cleavage site described above. This VNp may be expressed from the same vector as the vesicle nucleating polypeptide fused to the amino terminus of the biological molecule, or may be expressed from different vectors, for example plasmids or episome, or may be chromosomally expressed. It will be recognised that the protease will be selected for its activity and ability to be expressed in the selected host cell.

Biomolecules which can be employed in the present invention include, for example, carbohydrates, such as sugars; antigens; nucleic acids; and especially recombinant polypeptides.

Polypeptides, especially recombinant polypeptides, include therapeutic proteins and peptides, including cytokines, growth factors, antibodies, antibody fragments, immunoglobulin-like polypeptides, enzyme, vaccines, peptide hormones, chemokines, receptors, receptor fragments, kinases, phosphatases, isomerases, hydrolases, transcription factors and fusion polypeptides.

Antibodies include monoclonal antibodies, polyclonal antibodies and antibody fragments having biological activity, including multivalent and/or multi-specific forms of any of the foregoing.

Naturally occurring antibodies typically comprise four polypeptide chains, two identical heavy (H) chains and two identical light (L) chains inter-connected by disulphide bonds. Each heavy chain comprises a variable region (V_{H}) and a constant region (C_{H}), the C_{H} region comprising in its native form three domains, C_{H}1, C_{H}2 and C_{H}3. Each light chain comprises a variable region (V_{L}) and a constant region comprising one domain, C_{L}.

The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Antibody fragments which may be expressed comprise a portion of an intact antibody, said portion having a desired biological activity. Antibody fragments generally include at least one antigen binding site. Examples of antibody fragments include: (i) Fab fragments having V_{L}, C_{L}, V_{H} and C_{H}1 domains; (ii) Fab derivatives, such as a Fab' fragment having one or more cysteine residues at the C-terminus of the C_{H}1 domain, that can form bivalent fragments by disulfide bridging between two Fab derivatives; (iii) Fd fragment having V_{H} and C_{H}1 domains; (iv) Fd derivatives, such as Fd derivatives having one or more cysteine residues at the C-terminus of the C_{H}1 domain; (v) Fv fragments having the V_{L} and V_{H} domains of a single arm of an antibody; (vi) single chain antibody molecules such as single chain Fv (scFv) antibodies in which the V_{L} and V_{H} domains are covalently linked; (vii) V_{H} or V_{L} domain polypeptide without constant region domains linked to another variable domain (a V_{H} or V_{L} domain polypeptide) that is with or without constant region domains, (e.g., V_{H}-V_{H}, V_{H}-V_{L}, or V_{L}-V_{L}) (viii) domain antibody fragments, such as fragments consisting of a V_{H} domain, or a V_{L} domain, and antigen-binding fragments of either V_{H} or V_{L} domains, such as isolated CDR regions; (ix) so-called “diabodies” comprising two antigen binding sites, for example a heavy chain variable domain (V_{H}) connected to a light chain variable domain (V_{L}), in the same polypeptide chain; and (x) so-called linear antibodies comprising a pair of tandem Fd segments which, together with complementary light chain polypeptides, form a pair of antigen binding regions.

Preferably, the biological molecule or recombinant protein is not constitutively expressed to meaningfully detectable levels in an expression system in which the fusion protein of the present invention is expressed. For example, the recombinant protein may be a membrane binding protein, an insoluble protein, or a protein usually toxic to the host cell in which the fusion protein is expressed. In many embodiments, the biological molecule or recombinant protein is heterologous to the host cell.

The biological molecule need not be limited by size but may fall in the range of from less than 1 kDa up to 100 kDa. Examples of recombinant proteins include Akyrin Repeat Protein off7 (DARP), Stefin A, Uricase, Erythropoietin (EPO), Etanercept, FGF21, human growth hormone (hGH), DNAsel, and nanobodies. The size of the biological molecule to be exported is ultimately dependent on the size of vesicle that the host cell is able to produce. It is generally thought that vesicle sizes of 0.16-0.4 μm in diameter are the most likely and suitable for optimal export.

The present invention also resides in a nucleotide or vector (plasmid) sequence encoding, and especially an expression cassette expressing, the fusion protein described herein above. Suitable plasmids include plasmids from pUC19 and pBR322 based lineages and others, such as the plasmids described in international patent application WO2007/088371. It will be appreciated that the fusion proteins described herein above may be expressed from the same vector, or may be expressed from different vectors, for example plasmids or episome, or may be chromosomally expressed.

The nucleotide or vector sequence preferably employs a promoter, and examples of such promoters are well known in the art. The promoter may comprise a host cell polymerase promoter or a heterologous polymerase-dependent promoter, such as a phage polymerase-dependent promoter. Suitable promoters include CMV, hEF1, rhamnose, arabinose, lambda pL, T7A3, Tac and T7 based promoters. In certain embodiments, the nucleotides or vectors comprise E. coli polymerase-dependent promoters, particularly inducible promoters, such as those comprising one or more operators, such as lac operators, including perfect palindrome lac operator sequences. In many preferred embodiments, the nucleotides or vectors comprise lambda pL promoters operably linked to a single perfect palindrome lac operator sequence or a T7A3 promoter operably liked to two perfect palindrome lac operators sequences, one operator sequence located upstream of the promoter and one located downstream of the promoter. Most preferably, an operator overlaps the transcriptional start point.

Optionally, the nucleotide or plasmid sequence further comprises a leucine zipper sequence, such as that illustrated in SEQ ID NO:29.

The invention also resides in a host cell expressing the fusion protein or comprising the nucleotide sequence or plasmid as described herein. The host cell may be a prokaryotic cell such as bacteria, a single celled eukaryotic organism such as yeast, or a cultured eukaryotic cell line. Particular examples of a prokaryotic host cell include Escherichia coli strains, Bacillus strains (inc. subtillis and megatarium), Schwanella oneidensis bacteria. Suitable E. coli strains include BL21 DE3 and K12 lineage strains, as well as others (e.g. DH10b and JM109). Specific examples of prokaryotic cells include bacterial cells, for example gram-negative bacterial cells, including E. coli, Salmonella typhimurium, Schwanella oneidensis, Serratia marsescens and Pseudomonas aeruginosa, and gram-positive bacterial cells including Bacillus subtilis and megatarium. Examples of suitable cultured eukaryotic cell lines include HEK and CHO cell lines. Preferred host cells are bacteria, particularly enterobacteriacae, preferably E coli, and especially B or K12 strains thereof. In certain embodiments, the prokaryotic cell is engineered to be deficient in at least one native protease. In many preferred embodiments, the prokaryotic host cell is an ompT—E. coli strain, especially a W3110 E. coli strain.

Optionally, the host cell additionally expresses a VNp-leucine zipper peptide or a peptide comprising the VNp polypeptide described herein and a leucine zipper sequence such as that described herein. It will be appreciated that this additional peptide may comprise, include or have a sequence as set out in anyone of SEQ ID NOs:2-28. Alternatively, the host cell may include a nucleotide sequence that expresses a VNp-leucine zipper peptide as described herein or having a sequence as set out in SEQ ID NO:29.

In an embodiment, the host cell alternatively or additionally co-expresses one or more VNp tagged proteases in addition to a VNp-tagged target protein (VNp-tagged fusion protein) of the present invention and as described herein. In this way, proteolytic cleavage of the target protein from the VNp may be obtained within the vesicle. It will be appreciated that the co-expressed protease will be selected to be suitable for use with its particular protease cleavage site included in the fusion protein. Further processing of the target protein may additionally be required where the selected protease leaves some of the protease recognition site attached to the target protein.

The present invention also encompasses the use of an isolated vesicle nucleating polypeptide (VNp) fusion protein or nucleotide sequence as described herein to promote, enhance or increase the formation of vesicles within a host cell as part of a recombinant expression system.

Expressed in another way, the present invention also resides in a method for promoting, enhancing or increasing the formation of vesicles in a recombinant expression system by expressing a polypeptide comprising an isolated vesicle nucleating polypeptide (VNp), a fusion protein or a nucleotide sequence as described herein in a host cell.

The present invention also encompasses the use of an isolated vesicle nucleating polypeptide (VNp), a fusion protein, a nucleotide sequence or a host cell as described herein to produce soluble and functional biological molecules from a cellular expression system, wherein the biological molecule is not constitutively expressed in the host cell of the expression system.

The invention also encompasses the use of an isolated vesicle nucleating polypeptide (VNp), a fusion protein, a nucleotide sequence or a host cell as described herein to enhance or increase the yield of soluble and functional biological molecules from a cellular expression system, wherein the biological molecule is constitutively expressed in the host cell of the expression system.

The term “functional” is used to mean a biological molecule that is folded in such a way that the molecule is biologically active. Such folding may not necessarily be the same or to the same extent as is found in the natural molecule, providing the molecule has some intrinsic biological activity.

Expressed in a yet further way, the present invention resides in a method of enhancing the (overall) yield of soluble and functional forms of a biological molecule from a cellular expression system, wherein the biological molecule is not constitutively expressed in a host cell of the expression system and wherein the method comprises expressing the VNp, fusion protein or nucleotide sequence of the present invention as described herein in a host cell.

In one embodiment, the polypeptide, fusion protein or nucleotide sequence promotes, enhances or increases release of the vesicles from the host cell into culture media of the expression system.

Preferably, the method further comprises separating the polypeptide, fusion protein or a product expressed by the nucleotide sequence from culture media.

The host cell may be a host cell as described herein.

Formation of vesicles within the host cell may be further enabled, promoted or enhanced by acetylation of the VNp at the amino terminal of the polypeptide.

There is also contemplated the use of amino terminal acetylation of an isolated vesicle nucleating polypeptide (VNp) or a fusion protein as described herein to promote, enhance or increase the formation of vesicles in a host cell as part of a recombinant expression system, wherein acetylation optionally or additionally promotes the release of vesicles from the host cell into culture (growth) media.

Methods and uses of the invention may further comprise the additional expression of a VNp-leucine zipper polypeptide as described herein. The addition of a dimerising leucine zipper sequence between the VNp and biological molecule, as well as the optional further additional expression of a VNp-Leucine zipper polypeptide, enhances expression of otherwise difficult to express (e.g. toxic or insoluble) biological molecules.

Preferably, the host cells are cultured at 20° C. to 40° C., such as 25° C. to 37° C., ideally under aerobic conditions.

In addition, the methods and uses described herein may further comprise the step of storing the vesicles either within or isolated from the host cells of the recombinant expression system. Storage at about 4° C. is considered appropriate. Storage may be for up to at least six months.

The methods and uses of the invention may further comprise the step of biological molecule purification. In this way, the biological molecule encapsulated in the vesicles may be released and purified for further processing or use. Methods to release and purify biological molecules from membrane-bound structures such as lipid vesicles are well known and standard in the art. Methods tested and confirmed as suitable (but are not exclusive to) include sonication (simplest and most attractive due to minimal impact on vesicle contents), treatment with detergent, or osmotic shock. Alternatively, the biological purification includes cleavage of the biological molecule from the VNp within the vesicle before release from the vesicle and any additional purification as may be required. In an embodiment, the in-vesicle cleavage is effected by a suitable protease which is ideally a VNp tagged protease co-expressed with the VNp tagged target protein.

The present invention also encompasses the co-expression and transport of a biological molecule, such as a carbohydrate, an antigen, nucleic acid or a recombinant protein, in combination with an isolated vesicle nucleating polypeptide (VNp) as described herein, as cargo in membrane-bound vesicles. It will be appreciated that, in the context, vesicle formation is promoted or enhanced by the VNp.

The present invention also contemplates the use of a recombinant expression system, such as E. coli, for the production of one or more biological molecules expressed in and optionally released from a host cell, wherein the expression system comprises an isolated vesicle nucleating polypeptide, fusion protein, nucleotide sequence or host cell as described herein.

The present invention enables export from cells of cargoes in soluble form, notably in instances when intracellular expression of the cargoes, for example many instances of recombinant polypeptide expressed in prokaryotic hosts, such as E. coli, are produced in insoluble form.

The present invention will now be described in further detail with reference to the following non-limiting examples and figures in which:

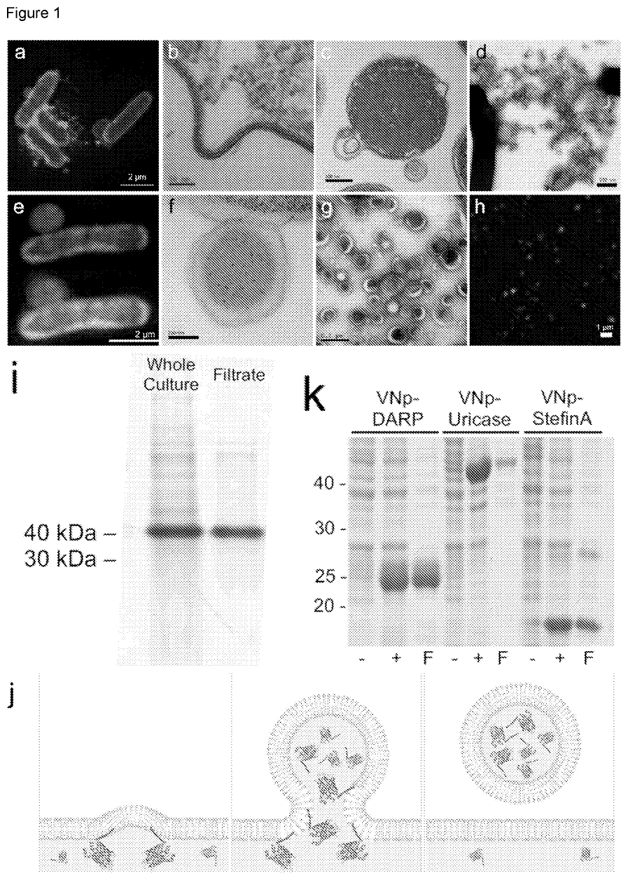

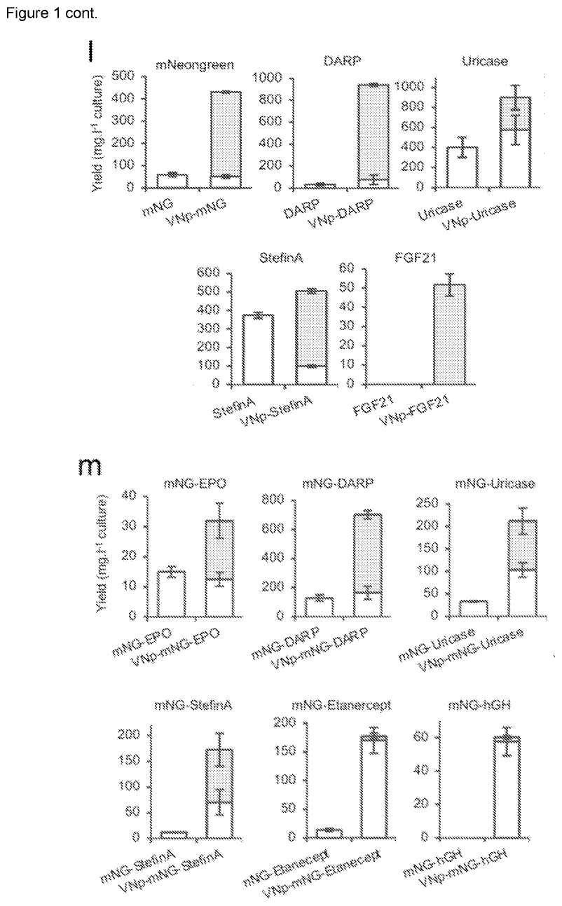

FIG. 1 Recombinant vesicle formation. FIG. 1a: OmpA-GFP SIM fluorescence; FIG. 1b and FIG. 1c: negative stained EM serial section images of VNp-induced membrane curvature in E. coli; FIG. 1d: EM of E. coli cells cultured on grids generating vesicles; FIG. 1e: mCherry (magenta) and -mNeongreen (green) SIM fluorescence of VNp2-mNeongreen OmpA-mCherry expressing E. coli cells; FIG. 1f: Anti-mNeongreen immuno-EM image of serial section though E. coli associated VNp-mNeongreen induced vesicle; FIG. 1g: EM images of isolated VNp-mNeongreen containing vesicles; FIG. 1h: fluorescence images of isolated VNp-mNeongreen containing vesicles; FIG. 1i: Coomassie stained gel of cell culture and filtered media of VNp2-mNeongreen expressing cells; FIG. 1j: Schematic of VNp-induced cargo containing vesicles; FIG. 1k: Coomassie stained samples of uninduced and induced cultures or filtered induced cultures of VNp2-DARP, VNp2-Uricase and VNp2-StefinA expressing cells; FIG. 1l: average yields per litre of culture for unlabelled mNeongreen labelled fusions of target proteins from cell extracts (empty columns) and media (filled columns); FIG. 1m: C-terminal mNeongreen labelled fusions of target proteins from cell extracts (empty columns) and media (filled columns). Errors are s.d. from ≥3 experimental repeats.

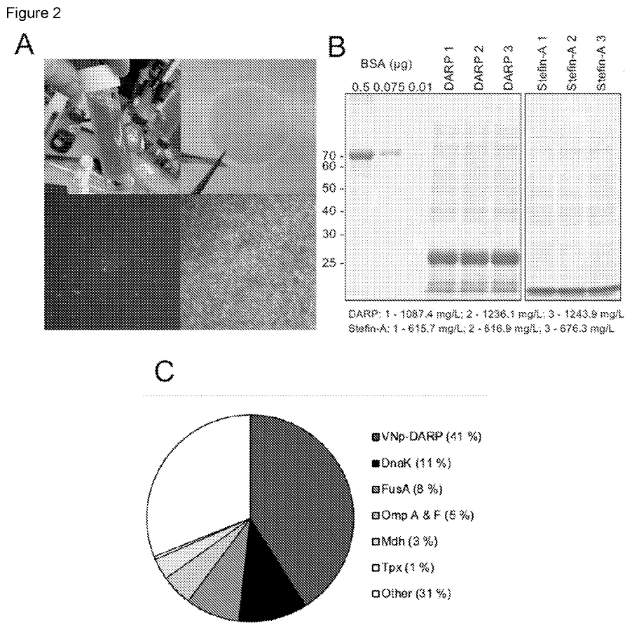

FIG. 2 Enrichment of VNp-fusions within the recombinant vesicles. FIG. 2A: Universal containing centrifuged media from an overnight culture of VNp-mNeongreen expressing cells (upper left) concentrated upon 0.1 μm filter (top right) and associated wide-field fluorescence images of mNeongreen containing vesicles. FIG. 2B: SDS-PAGE gel of BSA quantitation controls and 8 μl of centrifuged media fractions from VNp-fusion induction cultures from independent biological repeats. These samples were subsequently subject to tryptic digest and proteomic analysis. FIG. 2C: Pie chart showing typical composition of VNpfusion containing vesicles as determined from the proteomic analysis.

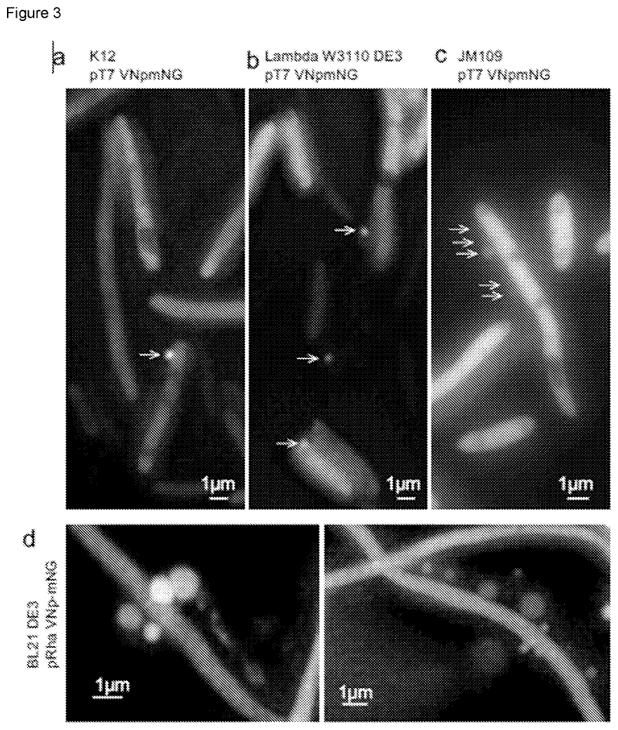

FIG. 3 VNp induced recombinant protein packaged vesicles using different E. coli strains and promoters. Widefield fluorescence images of (FIG. 3a) K12, (FIG. 3b) Lambda DE3 W3110, (FIG. 3c) JM109, and (FIG. 3d) BL21 DE3 E. coli strains expressing VNp-mNeongreen from the T7 (FIG. 3a, FIG. 3b, FIG. 3c) or Rhamnose (FIG. 3d) promoters.

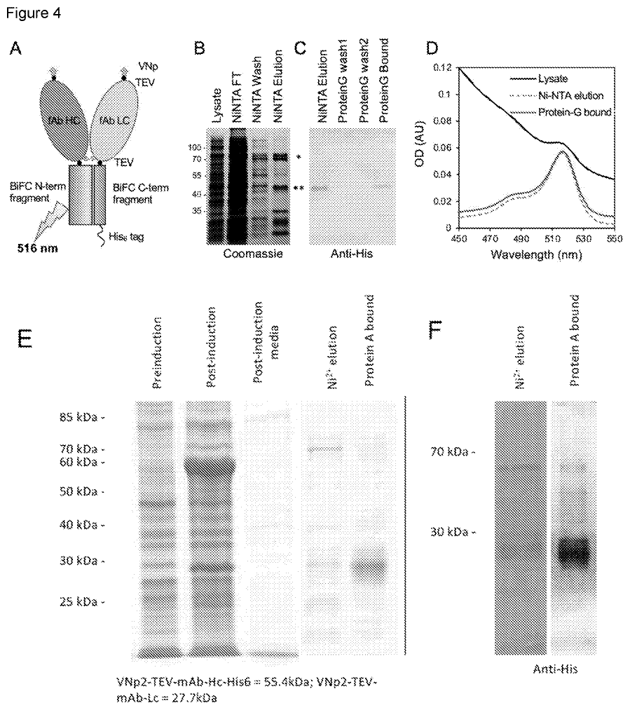

FIG. 4: The VNp tag permits expression and isolation of heterodimeric-soluble-functional antibodies from E. coli. FIGS. 4A-D: VNp-fAb purification from E. coli. FIG. 4A: Blmolecular-Fluorescence Complementation VNp-fAb fusions that allows detection of fAb Hc/Lc heterodimer formation. FIG. 4B: Stained SDS-PAGE and FIG. 4C: anti-His western blot (recognises HIS6 tagged Lc) of samples from VNp-Hc*/VNp-Lc** expression, nickel affinity purification and protein G binding. FIG. 4D: VNp-Hc/Lc heterodimer dependent BiFC fluorescence from cell lysate, nickel elution and subsequent Protein G bound fractions. FIGS. 4E-F: VNp-mAb expression and purification from E. coli. FIG. 4E: Stained SDS-PAGE and FIG. 4F: anti-His western blot (recognises HIS6 tagged Hc) of samples from VNp-mAB-Hc/VNp-mAB-Lc expression, nickel affinity purification and protein A binding.

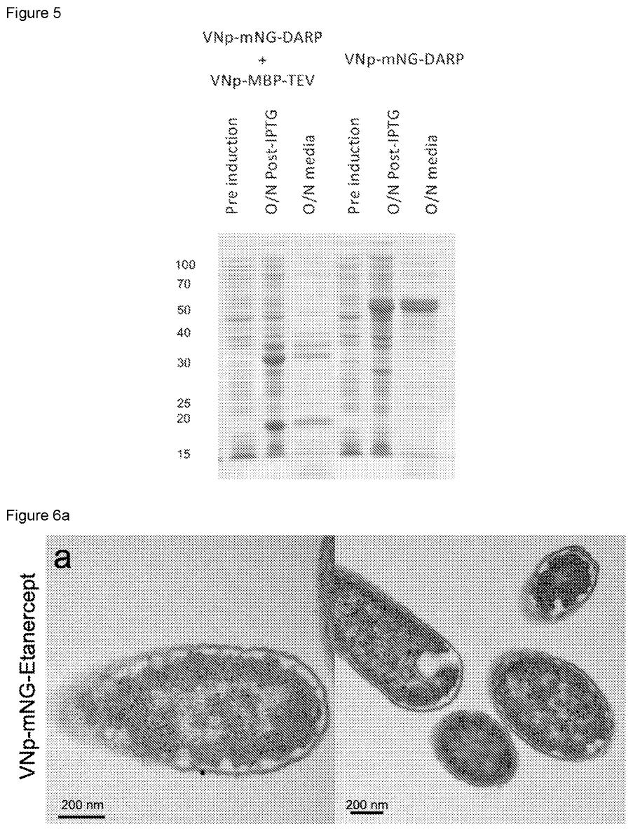

FIG. 5: VNps allow in vesicle targeted proteolytic cleavage of recombinant proteins. E. coli cells contained constructs to express a VNp-mNeongreen-DARPinoff7 fusion alone or in combination with VNp-Maltose Binding Protein (MBP)-TEV protease fusion were cultured and expression induced in an overnight flask culture. The VNp-mNeongreen-DARPinoff7 fusion contained a TEV cleavage site engineered between the mNeongreen and DARPin sequences. Expression of the VNp-mNG-DARP alone resulted in expression and vesicular export of full length protein (50.8 kDa size). In contrast co-expression of the same protein together with the TEV protease fusion resulted in cleavage of the VNp-mNG-DARP at the TEV site, and export of the subsequent export of the VNp-mNG (32 kDa) and DARPinoff7 (18.8 kDa) fragments into the exported vesicles within the culture media. Expected protein sizes: VNp-MBP-TEV—75.4 kDa, VNp-mNg-DARP—50.8 kDa; VNp-mNG—32 kDa; DARP—18.8 kDa.

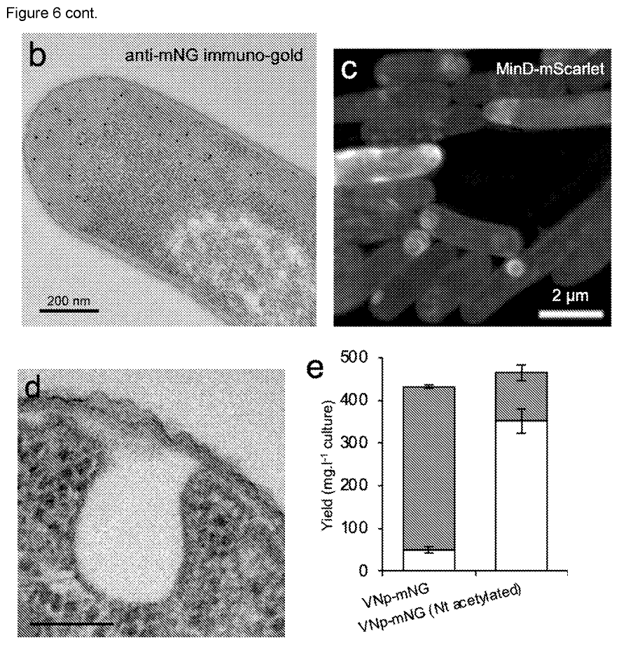

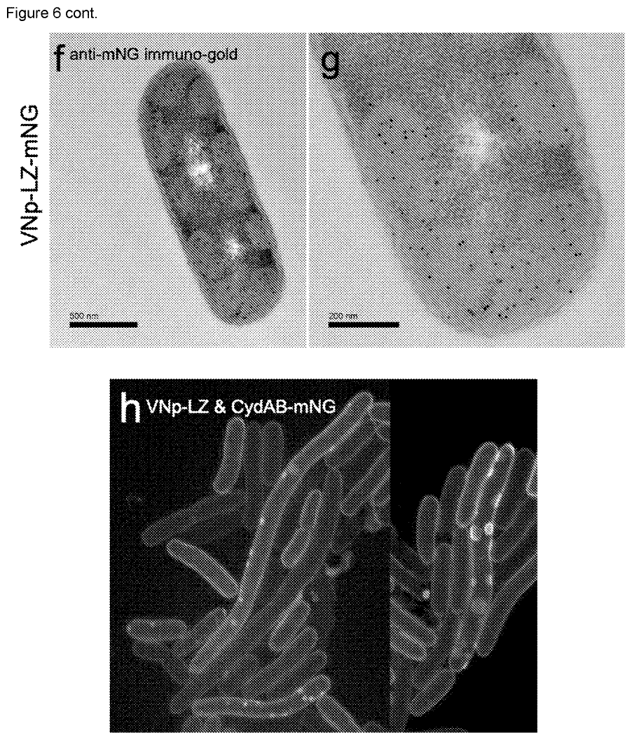

FIG. 6 VNp dimer fusions produce fusion containing cellular membrane packages. FIG. 6a: negative stained EM serial section images of VNp2-Etanercerpt induced inward membrane curvature in E. coli: FIG. 6b: anti-mNeongreen immuno-stained EM serial section images of VNp2-Etancerpt induced inward membrane curvature in E. coli. FIG. 6c: SIM imaging of MinD-mScarlet labelled inner membranes of E. coli co-expressing VNp2 and the NatB amino-terminal acetylation complex producing cytosolic membrane bound structures; FIG. 6d negative stained EM serial sections of E. coli co-expressing VNp2 and the NatB amino-terminal acetylation complex producing cytosolic membrane bound structures; FIG. 6e: amino terminal acetylation of VNp2-mNeongreen promotes mNeongreen incorporation within intracellular membrane bound structures; FIG. 6f and FIG. 6g: Anti-mNeongreen immuno-EM images of serial section though E. coli expressing VNp2-LZ-mNeongreen; FIG. 6h: SIM images of CydAB-mNeongreen labelled inner membranes in E. coli expressing VNp2-LZ show the VNp2-LZ dimer concentrates within the lumen of cytosolic inner membrane bound vesicles.

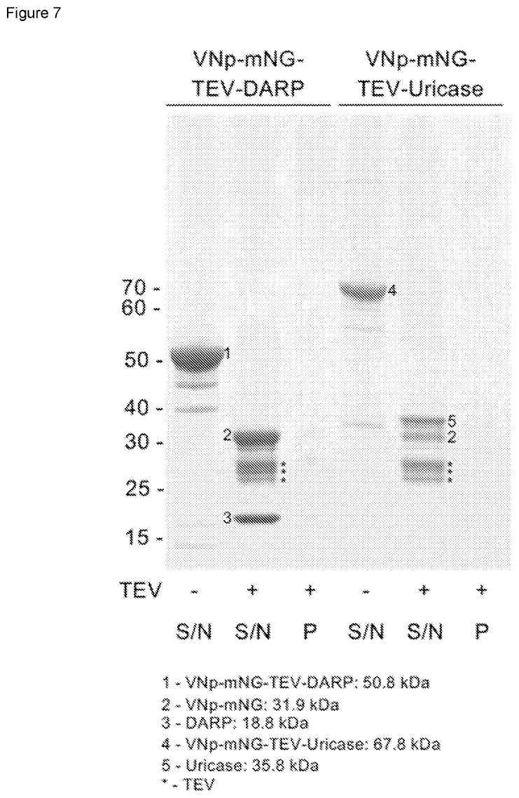

FIG. 7: VNp-fusions remain soluble after targeted protease cleavage of the VNp tag. Purified VNpmNG-TEV-DARP and VNp-mNG-TEV-Uricase were digested with TEV protease. The resultant cleaved proteins were not detectable within the pellet fraction (P) and remained in the supernatant (S/N) fraction after centrifugation at 13,200 RCF.

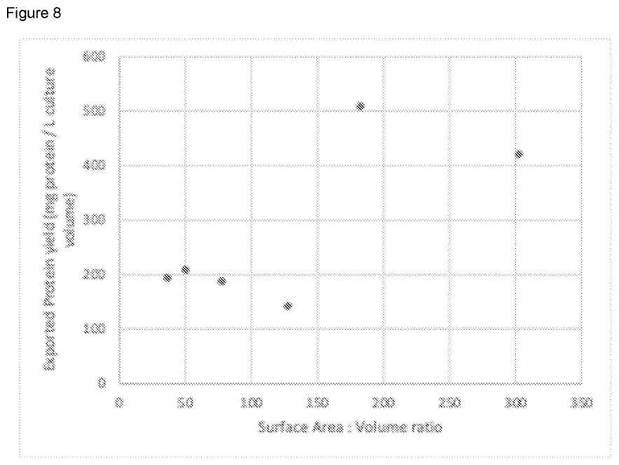

FIG. 8: VNp-fusions can be exported from cells grown in a range of culture volumes. Yields of exported VNp-mNeongreen were determined cultures of cells in a range of volumes, in different size flasks to modulate Surface Area to Volume ratio (X axis).

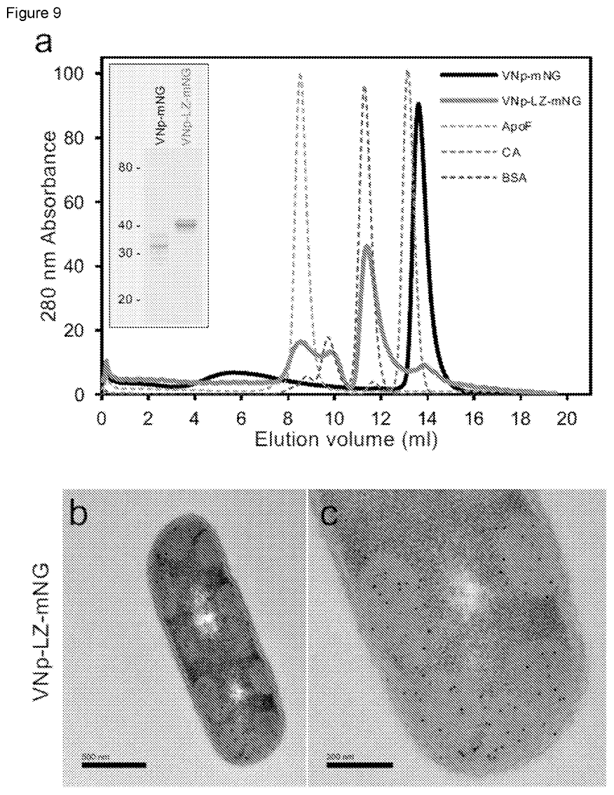

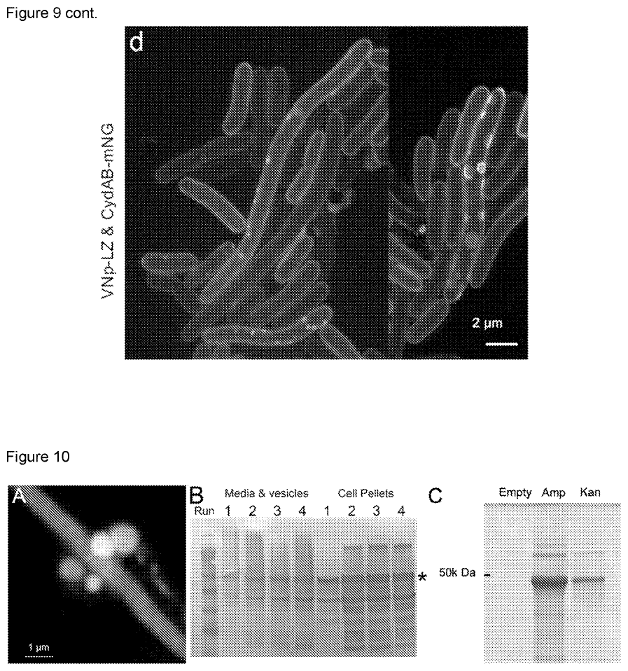

FIG. 9: Dimeric VNp-Leucine fusions lead to the formation of cytosolic VNp-fusion filled vesicles in E. coli. FIG. 9a: Size exclusion chromatography profiles of purified recombinant VNp-mNG and VNp-LZmNG proteins (inset) confirmed introduction of a Leucine Zipper (LZ) motif to the VNp-mNeongreen (mNG) fusion induced stable dimer formation. Each fusion protein as well protein standards (29 kDa carbonic anhydrase—blue; 66 kDa BSA—red; 443 kDa Apoferritin complex—yellow), were run using identical conditions. FIG. 9b and FIG. 9c: Anti-mNeongreen immuno-EM images of sections though E. coli expressing VNp-LZ-mNeongreen. FIG. 9d: SIM images of CydAB-mNeongreen labelled inner membranes in E. coli expressing VNp-LZ show the VNp-LZ dimer concentrates within the lumen of cytosolic inner membrane bound vesicles.

FIG. 10 Alpha-synuclein fusions induce vesicular export into the culture media when expressed in E. coli. FIG. 10a: Widefield image of E. coli expressing αSyn (60 amino acid sequence) fused to both mNeongreen and human growth hormone (αSyn-mNG-hGH) showing export of the αSyn fusion into extracellular vesicles. FIG. 10b: Anti-FLAG western of samples from bioreactor cultures of E. coli expressing αSyn-mNG-hGH shows expression and export to vesicles of the fusion protein (highlighted by *) in four independent experiments. FIG. 10c: SDS-PAGE analysis of filtered media from an overnight IPTG induction culture of E. coli containing either an empty expression vector or an ampilicillin or kanamycin resistance conferring vector allow expression of αSyn-mNG-hGH. αSyn-mNG-hGH is exported into the media independently of antibiotic selection.

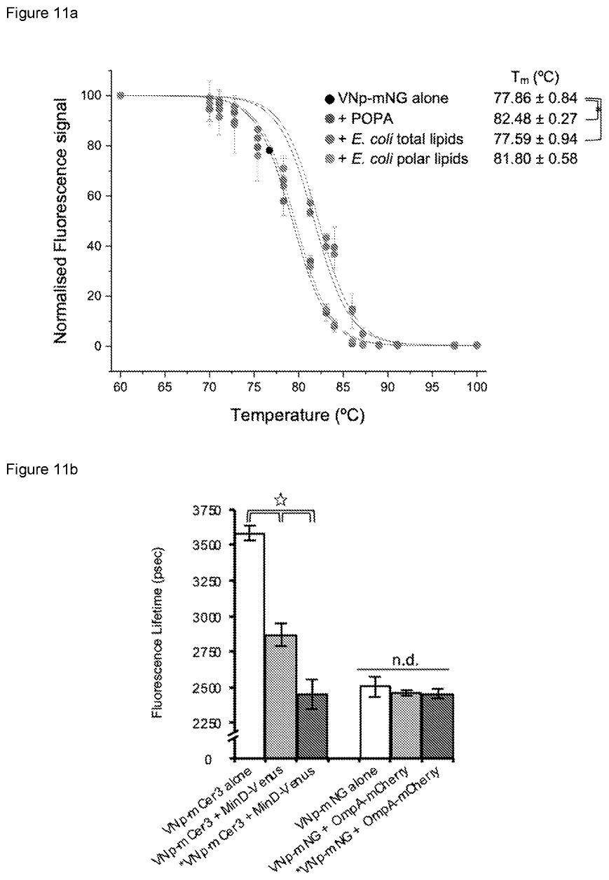

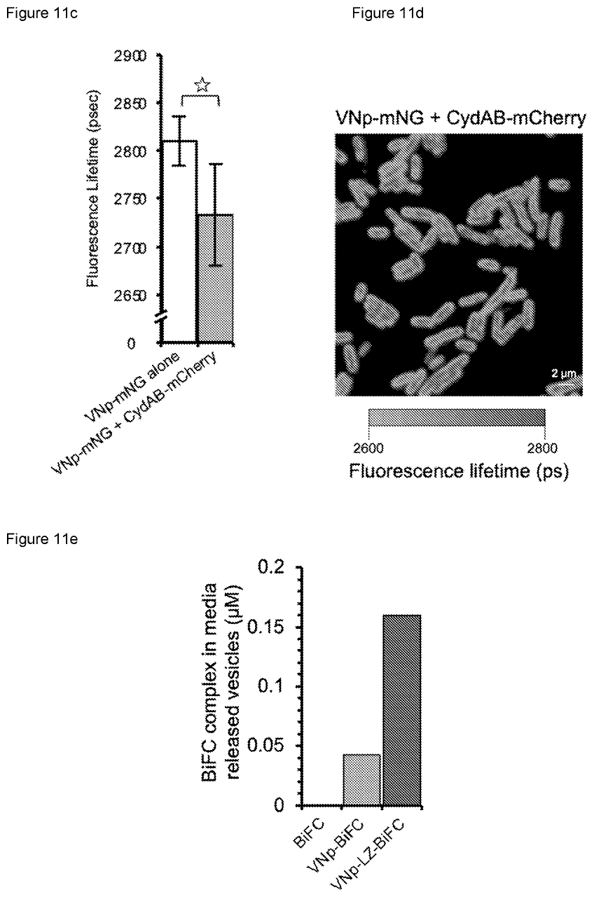

FIG. 11: VNp interacts with E. coli membranes in vivo and in vitro. FIG. 11a: Thermal shift mNeongreen fluorescence curve of VNp2-mNeongreen alone (black) or in the presence of 100 nm vesicles composed of phosphatidic acid (red), or mixtures of total (blue) or polar (green) E. coli lipids; FIG. 11b: Donor fluorophore Fluorescence lifetimes of E. coli cells expressing VNp donor fluorophore fusions (VNp2-Cer3/VNp2-mNG) with acceptor fluorophore labelled inner (MinD-Citrine) or outer (OmpA-mCherry) membrane proteins. *-Nt-acetylated VNp2; ⋆—different at 99.99% confidence levels. Data acquired using 60× water immersion objective; FIG. 11c: Histogram of mNeongreen fluorescence lifetime within E. coli cells expressing an mNeongreen donor fluorophore VNp fusion (VNp2-mNG), either alone or in combination with mCherry acceptor fluorophore labelled CydAB inner membrane complex. ⋆—different at 99.99% confidence levels; FIG. 11d: mNeongreen fluorescence Lifetime image of VNp2-mNG CydAB-mCherry expressing cells (from FIG. 10c) illustrating reduced donor lifetime at the cell membrane, where CydAB is located. FIGS. 10c and 10d: Acquired using 60× oil immersion objective. Reduction in lifetime length reflected in change from blue (2.8 ns) to green (2.6 ns); FIG. 11e: Circular Dichroism illustrating the alpha helical VNp structure is stabilised by E. coli lipid membranes. CD spectra of VNp2 (black), total E. coli lipid vesicles (grey) alone, or mixture of both VNp2 and E. coli lipid vesicles (red). The relative broad negative CD spectra peaks at 208 nm and 222 nm, observed in the mixture of VNp2 and E. coli lipid membrane are consistent with single α-helical structures, and show the VNp2 alpha helix is stabilised upon interaction with E. coli membrane lipid vesicles.

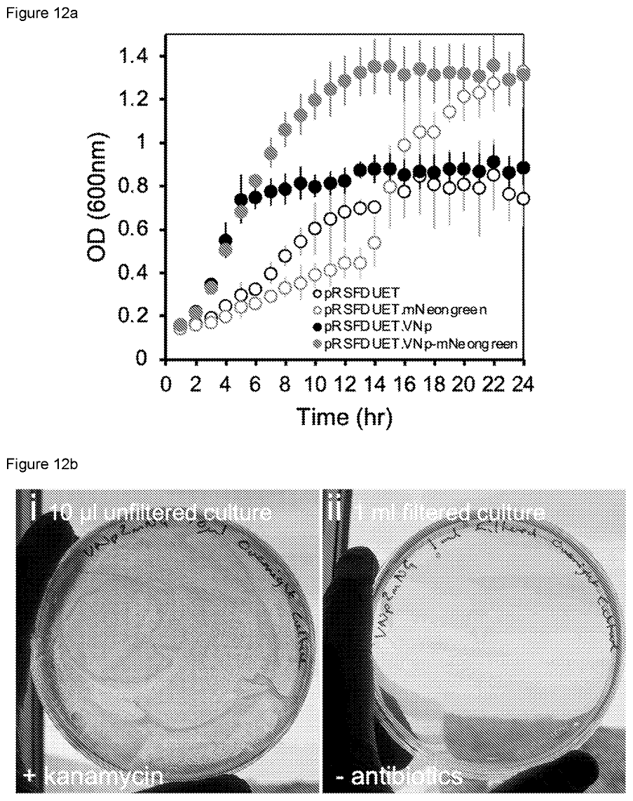

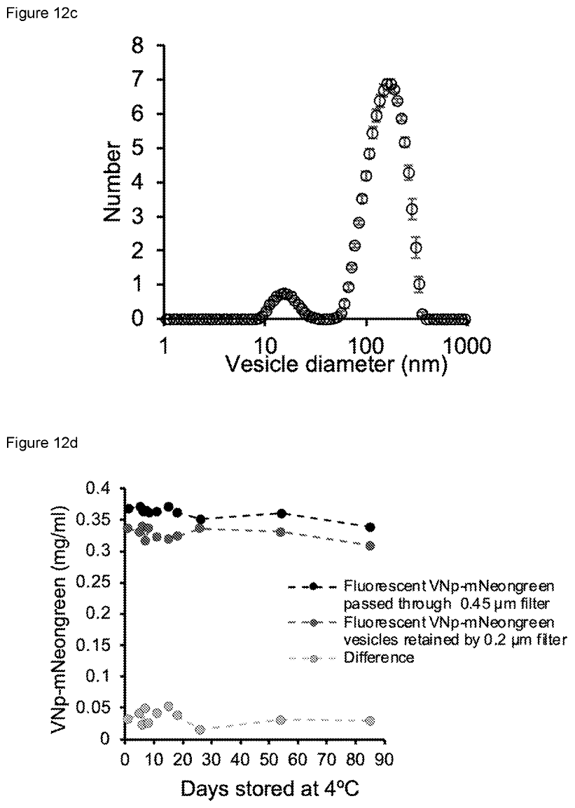

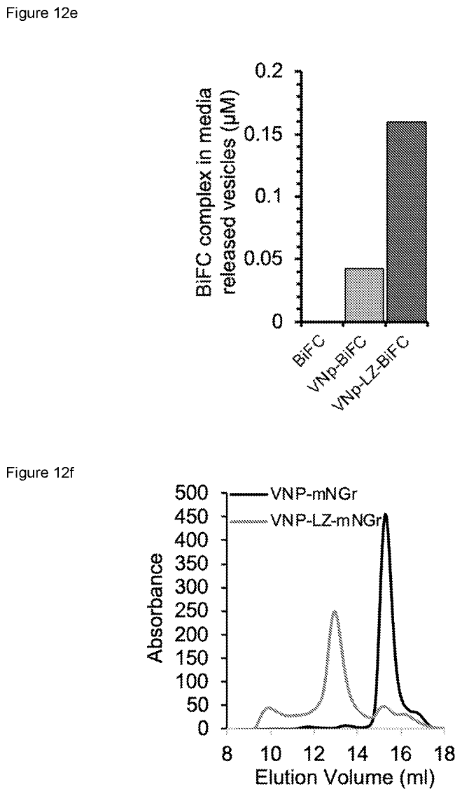

FIG. 12: FIG. 12a: Growth curves generated from averages from four replicate cultures of BL21(DE3) E. coli cells containing either empty pRSFDUET vector (empty black circles), pRSFDUET.mNeongreen (empty green circles), pRSFDUET.VNp2 (filled black circles) or pRSFDUET.VNp2.mNeongreen (filled green circles), grown at 37° C. in LB on the same 96-well plate, illustrate expression of VNp or a VNp fusion does not impact bacterial growth; FIG. 12b: Routine test illustrating exclusion of viable cells from the vesicle containing filtrate. 10 μl of total culture (i) and 1 ml of 0.45 μm media filtrate (ii) from an overnight culture of VNp2-mNG expressing E. coli cells were plated out onto LB (ii) or LB supplemented with kanamycin (i) and incubated overnight at 37° C.; FIG. 12c: Dynamic light scattering of filter purified VNp-mNeongreen induced vesicles. Graph presents averaged data (± standard errors) from ten separate analyses; FIG. 12d: VNp2-mNeongreen containing vesicles, filter purified from media of an overnight culture of BL21 DE3 pRSFDUET1-VNp2-mNeongreen cells were stored at 4° C. Overall mNeongreen fluorescence and the fraction of mNeongreen signal within 0.2 μm filter retained vesicles did not vary over time indicates stability of vesicles and folded mNeongreen protein; FIG. 12e: Fluorescence (515 nm) derived concentration of complexed Bimolecular Fluorescence Complementation fragments contained within vesicles isolated from BL21(DE3) E. coli expressing VenusN154 & VNp2-VenusC155 (white); VNp2-VenusN154 & VNp2-VenusC155 (grey); or VNp2-LZ-VenusN154_VNp2-LZ-VenusC155 (black); FIG. 12f: Gel filtration curves comparing elution profile of VNp2-mNG (black) and VNp2-LZ-mNG (grey) illustrate the leucine zipper induces dimerisation of the VNp fusion within vesicles.

FIG. 13: VNp fusion is contained within the lumen of isolated recombinant vesicles. VNp2-mNeongreen containing vesicles, filter purified from media of an overnight culture of BL21 DE3 pRSFDUET1-VNp-mNeongreen cells were mounted onto EM grids and subjected to anti-mNeongreen immuno EM analysis. mNeongreen dependent gold densities fragments were released from vesicles upon osmotic shock from resuspension in water. These were not observed in control samples subjected to either immuno-analysis prior to bursting, or without primary anti-mNeongreen antibodies.

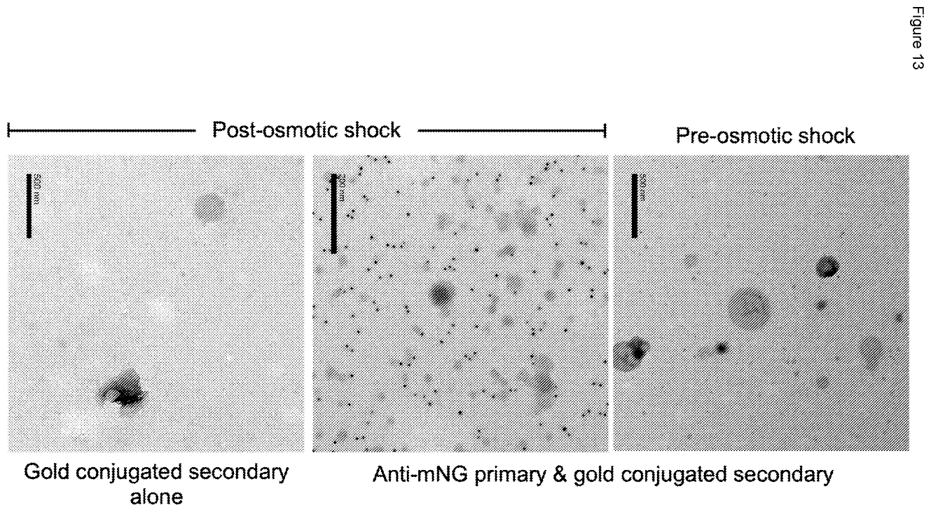

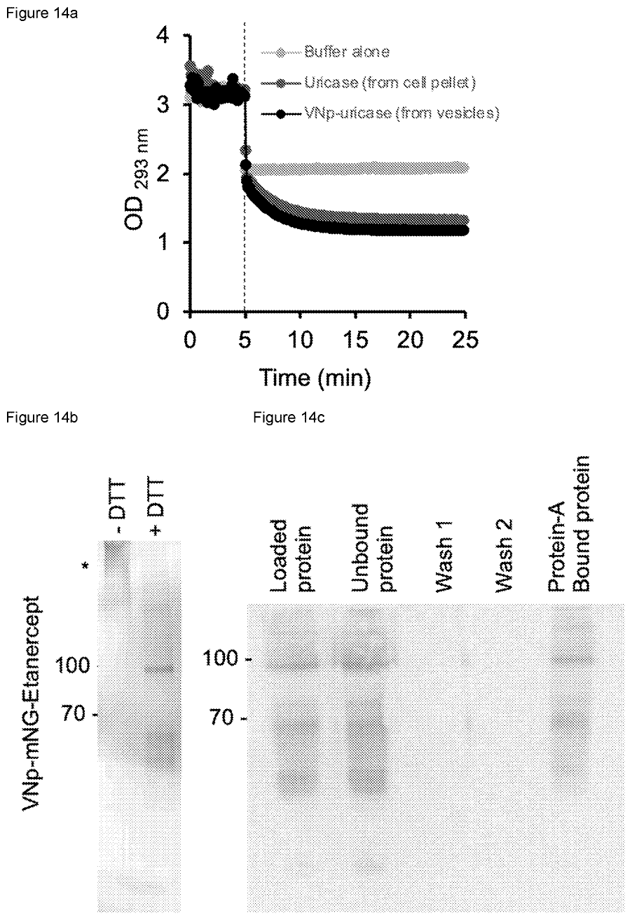

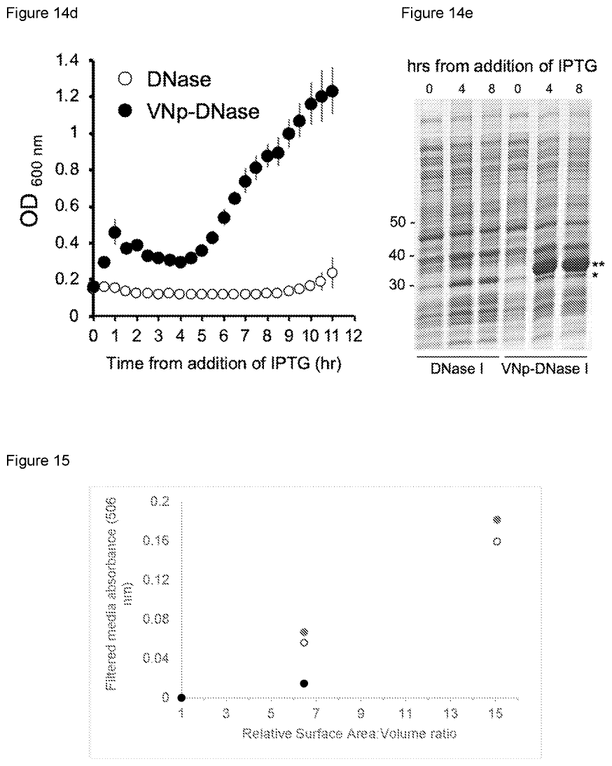

FIG. 14: Vesicle isolated VNp-fusions are functional. FIG. 14a: Uricase enzyme activity assay showing activity of Uricase that was either isolated from a cell pellet using conventional methods (red) or from VNp induced vesicles (black). A buffer only control (grey) shows dilution dependent change in baseline absorbance at 293 nm. Uricase enzyme/buffer was added to the substrate after a 5 min equilibration (dashed line); FIGS. 14b and 14c: Anti-mNeongreen western blots illustrating disulphide bond dependent oligomerisation (FIG. 6b) and functionality (FIG. 6c) of VNp2-mNG-Etanercept purified from E. coli; FIG. 14b: VNp2-mNG-Etanercept disulphide bond-dependent oligomers (*) are disrupted by addition of DTT reducing agent (left hand gel); FIG. 14c (Right hand gel) E. coli expressed affinity purified VNp-mNG-Etanercept-His₆ binds to Protein A—Dynabeads, which were subsequently washed in binding buffer, before being boiled in SDS-PAGE loading buffer, to release bound proteins. Predicted size of VNp-mNeongreen-Etanercept: 83.9 kDa; FIG. 14d: Growth curves and FIG. 14e: expression profile of E. coli expressing DNase and VNp2-Dnase (Predicted sizes of Dnase and VNp-Dnase are 30.2 and 34.4 kDa respectively).

FIG. 15: Export yields of VNp-mNeongreen containing vesicles from E. coli cultured in flasks of different sizes to vary surface area: volume ratios of the culture. Blue dots—standard Erlenmeyer flasks; red dots—baffled Erlenmeyer flasks; black dots—standard Erlenmeyer flasks sealed with parafilm to induce anaerobic conditions. Higher oxygenation resulted in increased vesicle export.

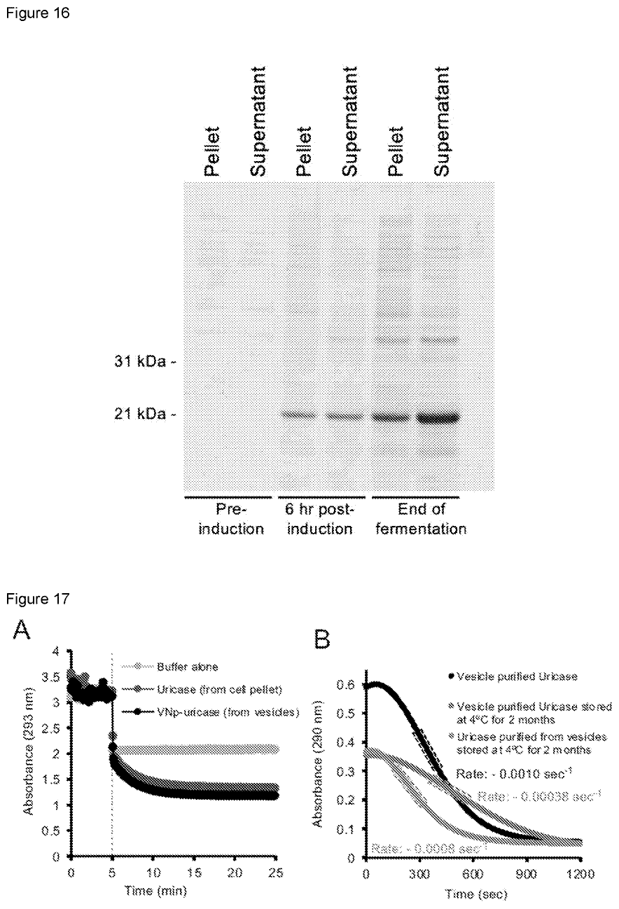

FIG. 16: Coomassie stained SDS-PAGE analysis of samples from example of 20 L fermenter E. coli VNp-DARPinOFF7 induction.

FIG. 17: VNp-fusion proteins remain active within vesicles when stored at 4° C. in sterile PBS/media for >6 months. FIG. 17a: The enzymatic activity of Uricase isolated from a cell pellet using conventional methods (red) or from VNp-uricase induced vesicles (black). A Grey=buffer only control. Uricase enzyme/buffer was added to the uric acid substrate after a 5 min equilibration (dashed line). FIG. 17b: stability of uricase enzyme activity from protein stored within VNp-uricase vesicles, uricase activity of either fresh vesicle purified VNp-uricase (black; the same vesicle purified VNp-uricase stored in reaction buffer at 4° C. for 2 months (green); or freshly purified from vesicles that had been stored at 4° C. for 2 months (blue), measured using stopped-flow. Rates were determined from steady state regions (highlighted by boxes) of averaged curves.

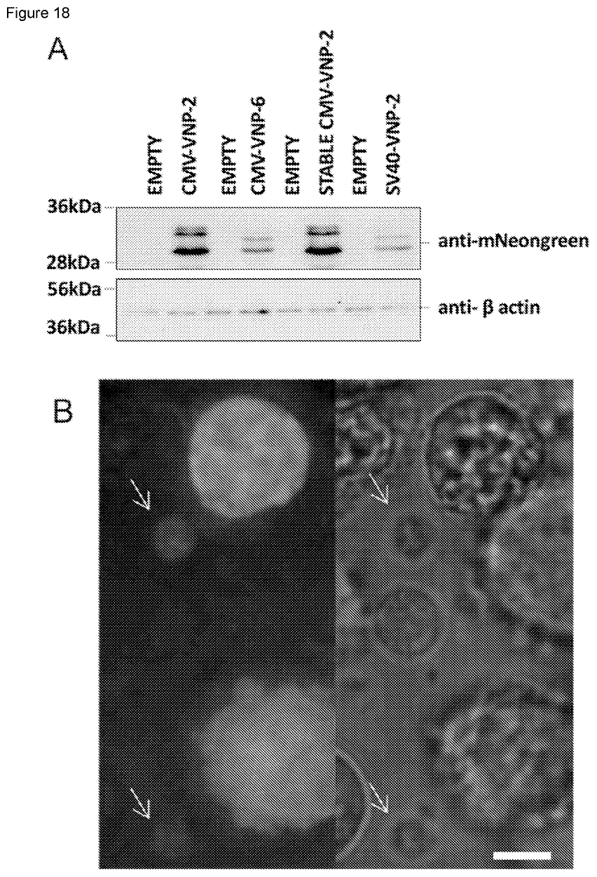

FIG. 18: Viability of VNp in mammalian cell lines. FIG. 18A: Mammalian HEK cell lines generated containing empty vectors, or plasmids containing either VNp2-mNeongreen or VNp6-mNeongreen under the control of CMV or SV40 promoters. FIG. 18B: HEK cells containing plasmids with VNp6-mNeongreen under the control of the SV40 promoter induced the formation of extracellular VNp6-mNeongreen containing vesicles (arrows).

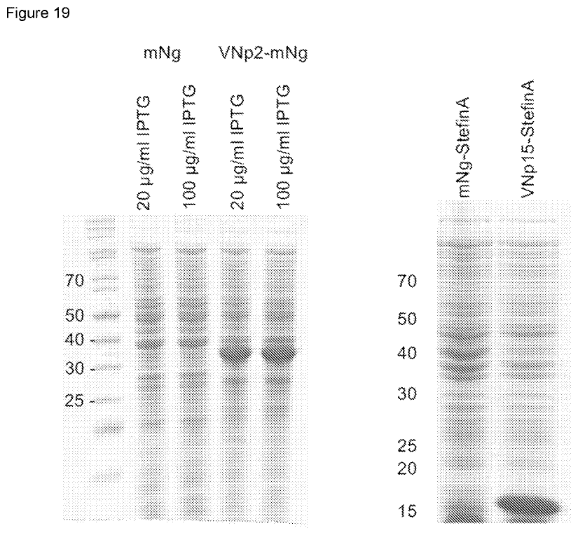

FIG. 19: VNp tag facilitates recombinant protein expression in anaerobic culture conditions. Gel showing expression profiles from cells containing expression constructs for mNeongreen and StefinA alone, or fused to a VNp. Expected protein sizes: mNg—28 kDa, VNp-mNg—32.5 kDa; mNg-StefinA—39 kDa; VNp-StefinA—14.5 kDa.

### Sequences

SEQ ID NO:1—Full length endogenous Hs alpha-synuclein (VNp1)

SEQ ID NO:2—Amino-terminal 38 aa of Hs alpha-synuclein (VNp2)

SEQ ID NO:3—Amino-terminal 38 aa of Hs beta-synuclein (VNp3)

SEQ ID NO:4—Amino-terminal 38 aa of Hs gamma-synuclein (VNp4)

SEQ ID NO:5—VNp2 with 3K-E mutations (VNp5)

SEQ ID NO:6—VNp4 with 2K-D mutations (VNp6)

SEQ ID NO:7—VNp4 with 6K-A mutations (VNp7)

SEQ ID NO:8—VNp4 with A29M mutation (VNp8)

SEQ ID NO:9—VNp6 with I10A mutation (VNp9)

SEQ ID NO:10—VNp6 with A11P.Q24P mutations (VNp10)

SEQ ID NO:11—VNp6 with E31K mutation (VNp11)

SEQ ID NO:12—VNp6 with D2G mutation (VNp12)

SEQ ID NO:13—VNp6 with D2P mutation (VNp13)

SEQ ID NO:14—VNp6—20 amino acid truncation (VNp14)

SEQ ID NO:15—VNp6—25 amino acid truncation (VNp15)

SEQ ID NO:16—VNp6—30 amino acid truncation (VNp16)

SEQ ID NO:17—VNp6—18 amino acid truncation (VNp17)

SEQ ID NO:18—VNp6—15 amino acid truncation (VNp18)

SEQ ID NO:19—VNp6—11 amino acid truncation (VNp19)

SEQ ID NO:20—VNp6—20 amino acid truncation with F4Y mutation (VNp20)

SEQ ID NO:21—VNp6—20 amino acid truncation with F4A mutation (VNp21)

SEQ ID NO:22—VNp6—20 amino acid truncation with D2P mutation (VNp22)

SEQ ID NO:23—VNp6—20 amino acid truncation with K6D mutation (VNp23)

SEQ ID NO:24—VNp22 with K5D and K6D mutations (VNp24)

SEQ ID NO:25—VNp6—10 amino acid truncation (VNp25)

SEQ ID NO:26—VNp6—9 amino acid truncation (VNp26)

SEQ ID NO:27—VNp14 with S9D mutation (VNp27)

SEQ ID NO:28—VNp2 with S9D mutations (VNp28)

SEQ ID NO:29—Leucine zipper sequence

The present invention relates to a method to program a simple cell to create membrane packages which can be filled with diverse molecules of interest. The method provides a way to produce target protein-filled membrane packages in vivo, but also sheds light on how an inducible synthetic post-translational modification regulates whether the package remains preferentially within the cytosol or is released out of the cell.

### Materials

E. coli strains used in this study:

| | |
|---|---|
| BL21 DE3 | F⁻ompT hsdS_{B} (r_{B}⁻, m_{B}⁻) gal dem (DE3). |
| DH10β | F⁻mcrA Δ(mrr-hsdRMS-mcrBC) |
| | φ80lacZΔM15 ΔlacX74 recA1 endA1 |
| | araD139 Δ(ara-leu)7697 galU galK λ⁻ rpsL(Str^{R}) nupG |
| W3110F⁻ | λ⁻ IN(rrnD-rrnE)1 rph-1 |
| CLD1040 | F⁻ λ, IN(rrnD-rrnE)1 rph-1 OmpT |
| JM109 | F⁻ traD36 proAB laql^{q}ZΔM15 endA1 recA1 |
| | gyrA96 thi hsdR17 (rₖ⁻, mₖ⁺) relA1 |
| | supE44 Δ (lac-proAB) |

### Plasmids

pRSFDuet-1_VNp-His6; pRSFDuet-1_VNp-mNeongreen; pRSFDuet-1_VNp-mNeongreen_OmpA-mCherry; pRSFDuet-1_VNp-mNeongreen_mScarlet-minD; pDuet-1_VNp-mCerulean3; pDuet-1_VNp-mNeongreen; pDuet-1_VNp-mNeongreen_VNp-mCherry; pDuet-1_VNp-mNeongreen_VNp-mCherry; pDuet-1_VNp-mCerulean3_Citrine-minD; pACYCDuet-1_naa20_naa25 (pNatB) (Johnson, M. et al (2010) PLoS ONE 5, e15801); pRSFDuet-1_VNp-LZ-mNeongreen; pRSFDuet-1_DARPinOFF7; pRSFDuet-1_VNp-DARPinOFF7; pRSFDuet-1_Uricase; pRSFDuet-1_VNp-Uricase; pRSFDuet-1_StefinA; pRSFDuet-1_VNp-stefinA; pRSFDuet-1_FGF21; pRSFDuet-1_VNp-FGF21;pRSFDuet-1_hGH: pRSFDuet-1_VNp-hGH: pRSFDuet-1_VNp-LZ-hGH; pRSFDuet-1_mNeongreen; pRSFDuet-1 mNeongreen-DARPinOFF7: pRSFDuet-1 VNp-mNeongreen-DARPinOFF7;pRSFDuet-1_mNeongreen-Uricase; pRSFDuet-1_VNp-mNeongreen-Uricase; pRSFDuet-1_mNeongreen-StefinA; pRSFDuet-1_VNp-mNeongreen-StefinA; pRSFDuet-1_mNeongreen-Etanercept; pRSFDuet-1_VNp-mNeongreen-Etanercept; pRSFDuet-1_mNeongreen-Erythropoietin; pRSFDuet-1_VNp-mNeongreen-Erythropoietin; pRSFDuet-1_VNp-LZ_VNp-LZ-Etanercept; pRSFDuet-1_VNp-LZ_VNp-LZ-hGH.

Plasmid sequences and constructs deposited at addgene.org.

Uniprot accession numbers of cargo full length protein sequences tested in this study:
DARP—Akyrin Repeat Protein off7 (Agrobacterium radiobacter)—B9JMD9
DNasel—Deoxyribonuclease I (Bos taurus)—P00639
EPO—Erythropoietin (Homo sapiens)—P01588
Etanercept—Tumor necrosis factor receptor superfamily member 1B (Homo sapiens)—P20333
FGF21—Fibroblast Growth Factor 21 (Homo sapiens)—Q9NSA1
hGH—Somatotrophin (Homo sapiens)—P01241
mNeongreen (Branchiostoma lanceolatum)—A0A1S4NYF2
StefinA—Cystatin-A (Homo sapiens)—P01040
Uricase (Cyberlindnera jadinii)—P78609

### Methods

Bacterial cell culture and protein induction: All bacterial cells were cultured at 37° C. using Lysogeny Broth (10 g Tryptone; 10 g NaCl; 5 g Yeast Extract (per litre)) and Terrific Broth (12 g Tryptone: 24 g Yeast Extract: 4 ml 10% glycerol; 17 mM KH₂PO4 72 mM K₂HPO₄ (per litre) media. 5 ml LB starters from fresh bacterial transformations were cultured at 37° C. to saturation and used to inoculate 100-500 ml volume TB media, flask cultures that were incubated overnight at 37° C. with 200 rpm orbital shaking. Recombinant protein expression from the T7 promoter was induced by addition of isopropylthio-β-galactoside (IPTG) to a final concentration of 20 μg/ml (except etanercept where 10 μg/ml was used) once the culture had reached an OD₆₀₀ of 0.8-1.0). To generate amino-terminally acetylated VNp, target constructs were co-transformed into E. coli with pNatB, to allow co-expression with the fission yeast amino-α-acetyltransferase complex B, Naa20 and Naa25 (Johnson, M. et al (2010) PLoS ONE 5, e15801). Amino-terminal acetylation was confirmed by electrospray mass spectroscopy of the purified VNp fusion protein.

Growth curves were generated from 96 well plate cultures, prepared from late log-phase cultures, diluted into fresh media to an OD₆₀₀ of 0.1 nm at the start of the growth analysis experiment. OD₆₀₀ absorbance values were obtained using a Thermo Scientific Multiscan Go 1510-0318C plate reader and recorded using the Skanlt Software 4.0. OD₆₀₀ values were taken every 15 minutes for the duration of the experiment, and growth curves generated from averages of four individual biological repeats.

Soluble protein extracts: Cell pellets from 50 ml of culture were resuspended in 5 ml of soluble extract buffer (20 mM tris (hydroxymethyl) aminomethane (TRIS), 500 mM NaCl, pH 8.0), sonicated for a total of 2 min (6×20 sec pulses), and cell debris removed by centrifugation at 18,000 rpm (4° C.) for 30 min. Target protein concentration was determined using fluorescence of mNeonGreen fusion or gel densitometry. Both techniques were compared directly on the same samples to determine equivalence.

Recombinant Vesicle isolation: Vesicles were isolated directly from bacterial cell cultures by passing the culture through a sterile and detergent-free 0.45 μm polyethersulfone (PES) filter. Typical purity and concentration from equivalent volume of culture and filter flow through are shown in FIG. 1. Exclusion of viable cells from the vesicle containing filtrate was routinely tested by plating onto LB plates lacking antibiotics and incubating overnight at 37° C. (example shown in FIG. 12)

Protein concentration determination: Fluorescence scan was used to determine the concentration of mNeongreen labelled proteins in vesicle-containing media and soluble protein extracts. Absorbance was measured at 506 nm using a Varian Cary® 50 Bio UV-Vis spectrophotometer, with measurements from an equivalent empty vector culture used for baseline correction, and concentration determined using an extinction coefficient of 116,000 M⁻¹cm⁻¹. Concentration of non-mNeongreen labelled proteins was determined by gel densitometry analysis of triplicate samples run alongside bovine serum albumin (BSA) loading standards on Coomassie stained SDS-PAGE gels. Gels were scanned and analysed using Image J software. Concentration was determined by both UV and densitometry for three independent VNp-mNeongreen samples to confirm parity between analysis techniques. Average yields in FIG. 1 and Table 2 were calculated (mg soluble target protein/litre culture) from a minimum of three independent biological repeats from cultures of BL21 DE3 E. coli cells grown in TB media.

Protein isolation from vesicles: Purified VNp induced vesicles were resuspended in ice cold 1× PBS before being sonicated to disrupt vesicle membrane and release the VNp-fusion protein. To purify carboxyl His₆ tagged recombinant VNp-fusion protein (all recombinant proteins expressed during this study contain carboxyl-terminal His₆ affinity tags) further, this solution was then mixed in a 1 in 5 dilution of 5× binding buffer (250 mM TRIS 2.5 M NaCl 5% Triton-X 50 mM Imidazole pH 7.8) before passing over a Ni²⁺-agarose resin gravity column. Cytosolic recombinant protein was purified by passing soluble protein extracts (supplemented with Imidazole to 20 mM) over the Ni²⁺-agarose resin gravity column. In both cases matrix bound His-tagged protein was washed, eluted (using imidazole), and dialysed into appropriate storage or assay buffer. Protein identity and amino-terminal acetylation of isolated proteins was confirmed by electrospray mass-spectroscopy.

Determination of cytosolic VNp concentration: VNp-mNeongreen expression was induced in BL21 DE3 E. coli for 4 hours at 37° C. (when extracellular vesicle production is observed), and images of more than 120 cells were acquired using widefield imaging (described below) from 3 independent sample preparations. Mean mNeongreen intensity was determine from a 5×5 pixel area within each E. coli cell. A calibration line was generated from multiple images of known concentrations of slide-mounted VNp-mNeongreen solutions using identical imaging conditions as cell image acquisition. This was used to calculate the average cytosolic mNeongreen concentration from the total average intensity from all cells measured (18.76±0.14 μM).

Affinity purification of His-tagged proteins: To isolate recombinant protein from vesicles. media from an induced overnight culture expressing VNp-labelled protein was passed through a 0.45 μm polyethersulfone (PES) filter, and the subsequent vesicle containing flow-through was sonicated and mixed in a 1 in 5 dilution of 5× binding buffer (250 mM TRIS 2.5 M NaCl 5% Triton™-X 50 mM Imidazole pH 7.8) before passing over a Ni²⁺-agarose resin gravity column. Cytosolic recombinant protein was purified by passing soluble protein extracts (supplemented with Imidazole to 20 mM) over the Ni²⁺-agarose resin gravity column.

In both cases matrix bound His-tagged protein was washed, eluted (using imidazole), and dialysed into appropriate storage or assay buffer. Protein identity and amino-terminal acetylation of isolated proteins was confirmed using electrospray mass-spectroscopy.

Circular Dichroism (CD): Measurements were made in 2 mm quartz cuvettes using a Jasco 715 spectropolarimeter. VNp protein and 100 nm extruded vesicles were diluted in CD buffer (10 mM potassium phosphate, 5 mM MgCl₂ pH 7.0) to a concentration of 0.4 mg/ml and 0.2 respectively. Broad negative peaks at 208 and 222 nm and a positive peak at <200 nm are consistent with an a-helical structure.

Electrospray LC-MS of proteins: Electrospray mass spectra were recorded on a Bruker micrOTOF-Q II™ mass spectrometer. Samples were desalted on-line by reverse-phase HPLC on a Phenomenex® Jupiter® C4 column (5 μm, 300 Å, 2.0 mm×50 mm) running on an Agilent® 1100 HPLC system at a flow rate of 0.2 ml/min using a short water, acetonitrile, 0.05% trifluoroacetic acid gradient. The eluant was monitored at 214 nm and 280 nm and then directed into the electrospray source, operating in positive ion mode, at 4.5 kV and mass spectra recorded from 500-3000 m/z. Data was analysed and deconvoluted to give uncharged protein masses with Bruker's Compass Data Analysis software.

In-gel tryptic digest and proteomic analysis of recombinant vesicles: Samples of purified VNp-DARP induced vesicles (shown in FIG. 7) were run on SDS-PAGE, which was subsequently Coomassie stained, and the whole sample lane cut out, cut into small pieces, which were subsequently transferred to a 1.5 ml microfuge tube and stored in distilled water at 4° C. until processing.

The gel particles were washed with 150 μl of freshly made 50 mM NH₄HCO₃: acetonitrile (1:1 ratio) for 15 mins. Liquid was removed and gel fragments resuspended in 150 μl acetonitrile for 15 mins, before liquid was again removed, and gel pieces were resuspended in 100 μl of 10 mM DTT in 50 mM NH₄HCO₃, and incubated for 30 min. at 56° C. Gel pieces were centrifuged, and excess liquid removed before incubating for 1 min with 100 μl of acetonitrile, which was again removed and gel fragments were suspended in 100 μl of 55 mM chloroacetamide in 50 mM NH₄HCO₃ and incubated for 20 min at room temp in the dark. Pellets were then centrifuged, and the chloroacetamide solution was removed. Gel pieces were subject to subsequent 15 min washes in 150 μl of 50 mM NH₄HCO₃: acetonitrile (1:1), and then150 μl of 50 mM NH₄HCO₃ for 15 min, and liquid was removed by centrifugation after each wash. Gel pieces were then washed for 15 mins with 200 μl of acetonitrile, and then rehydrated in 50 μl of digestion buffer (12.5 mM NH₄HCO₃, 10% acetonitrile) containing 5 ng/μl of trypsin, which was left overnight at room temperature.

Upon completion of digestion, 15 μl acetonitrile was added to the sample, which was then sonicate in an ultrasound bath for 15 mins. Gel fragments were isolated by centrifugation and the supernatant collected in a fresh 0.5 ml microfuge tube (A). The gel fragment pellet was resuspended in 30 μl 50% acetonitrile with 5% formic acid, and sonicate in an ultrasound bath for 15 mins, and pellet again isolated by centrifugation and supernatant collected in a fresh 0.5 ml microfuge tube (B). Contents of tube A and B were combined, vacuum dried, and subsequently resuspended in 20 μl of 5% acetonitrile, 0.1% TFA. Samples were run through Pierce C18 Spin Tips and analysis by nano-LCMS.

Recombinant protein expression in E. coli grown in anaerobic conditions: An air tight anaerobic culture vessel contained LB media supplemented with antibiotic and either 50 mM sodium fumarate or 100 mM Sodium nitrate (fumarate and nitrate acts as electron acceptor in place of oxygen). Oxygen was purged from the media and air-tight culture vessel for 20 mins with argon gas. Fresh BL21 DE3 E. coli transformation containing the expression construct used to inoculate 5 ml overnight culture in LB+ selection antibiotic. A 1:50 dilution of this starter culture was added (using a syringe and needle) to the oxygen purged culture vessel. This anaerobic culture was incubated at 37° C. with shaking for 2 hrs, when T7 promoter dependent protein expression was induced by the addition of oxygen purged IPTG (20 μg/ml final concentration). Cells were subsequently cultured for 24 hrs at 37° C., before harvesting and analysis.

Gel filtration assay: 500 μl of protein samples were loaded to a Superdex® 200 Increase 10/300 GL size-exclusion column (GE Healthcare Life Sciences) equilibrated at room temperature in phosphate-buffered saline (PBS) and run at 0.75 ml/min flow rate. Eluted proteins were measured by Viscotek® Sec-Mals 9 and Viscotek® RI detector VE3580 (Malvern Panalytical). Data was analysed using OmniSEC® software.

Lipid binding Assay: Affinity of VNp for E. coli membrane lipids was established using a thermal shift fluorescence binding assay adapted from Nji, E. et al (Nat. Commun. (2018) 9, 4253-12). Equivalent assay samples made up of: 65 μl 3 mg/ml of VNp-mNeongreen, 65 μl 1 mM of 100 nm extruded vesicles composed of the lipid mixture to be tested; 15 μl, 10% N-Octyl-β-D-glucopyranoside (OGP); and 5 μl 20 mM Tris-HCl PH 7.0, were prepared in PCR tubes and held at the defined temperature in a gradient PCR machine for 10 minutes. Samples were centrifuged at 18.000 xg, and supernatant fluorescence was determined in black 96 well plates (BRAND®, Germany) using a BMG Clariostar® (BMG Labtech). Fluorescence readings were normalised and used to create a melting curve, where the melting temperature (Tm) was determined using Origin software (OriginLab). The final Tm value was an average (±s.d) calculated from three independent sample repeats.

Uricase Assay: 500 μl of 100 mM Tris ph8.5 with 200 mM Uric acid was placed in a cuvette and OD₂₉₃ measurements were taken over for 4 or 5 minutes. Subsequently either 500 μl of 4.2 mg/ml purified VNp2-Uricase (dialysed into 0.1M Tris ph8.5) or dialysis buffer alone was added to the cuvette and OD₂₉₃ measurements taken for 25 minutes (adapted from Huang, S.-H. & Wu, T.-K. (2004) Eur. J. Biochem. 271, 517-523).

Widefield Fluorescence Microscopy: Cells were mounted onto coverslips under <1 mm thick circular LB-agarose (2%) pads, and attached with appropriate spacers onto glass slides, before being visualised on an inverted microscope (Mulvihill, D. P. (2017) Cold Spring Harb. Protoc. 2017, 761-773). Live cell imaging for each sample was completed within 30 mins of mounting the cell sample onto coverslips.

Structured Illumination Microscopy (SIM) was undertaken using a Zeiss Elyra PS. 1 microscope with a 100× NA 1.46 oil immersion objective lens (Zeiss α Plan-Apochromat) as described previously (Periz, J. et al (2019) Nat. Commun. 10, 1-16; Qiu, H. et al (2016) Science 352, 697-701). Briefly, cells were mounted under thin LB-agarose pads onto high precision No. 1.5 coverslips (Zeiss, Jenna, Germany). 488 nm and 561 nm laser were used to illuminate mNeongreen and mCherry/mScarlet fusions, respectively. The optical filter set consisted of laser blocking filter MBS 405/488/561 as the dichroic mirror, and the dual-band emission filter LBF-488/561. The total of three rotations of the illumination pattern were implemented to obtain two-dimensional information. Super-resolution SIM image processing was performed using the Zeiss Zen software. Two colour images were aligned using the same software following a calibration using pre-mounted MultiSpec™ bead sample.

Fluorescence Lifetime Imaging Microscopy (FLIM): The one- and two-photon systems used in this work have been previously described (Botchway, S. W. et al (2015) J. Microsc. 258, 68-78). Prior to FLIM data acquisition, protein expression levels were verified using confocal microscope. Here, a Nikon Eclipse C2-Si confocal scan head attached to an inverted Nikon TE2000 or Ti-E microscope was used. mNeongreen and mCherry FP were excited at 491 nm (emission 520/35 nm) and 561 nm (emission 630/50 nm) respectively using an NKT super continuum laser. FLIM images were obtained as follows: 2 photon (950 nm) wavelength light was generated by a mode-locked titanium sapphire laser (Mira F900, Coherent Laser Ltd), producing 180 fs pulses at 76 MHz. This laser was pumped by a solid-state continuous wave 532 nm laser (Verdi 18, Coherent Lasers Ltd). Fluorescence was collected through a BG39 filter for the donor fluorophore. The acceptor was not excited.

For one photon excitation FLIM, the system was equipped with a SuperK EXTREME NKT-SC 470-2000 nm supercontinuum laser (NKT Photonics) which generates at 80 MHz repetition rate with 70 ps pulse width. The desired wavelengths were selected using a SuperK SELECT 29 multi-line tunable filter (NKT photonics). Images were collected through either a 60× 1.2 NA water immersion (FIG. 11b and FIG. 14b) or 60× 1.49 NA oil immersion (FIG. 11c) lens. For both one and two-photon excitation, emission was collected by the same objective through filters (above) and detected with an external hybrid GaAsP (HPM-100-40, Becker & Hickl, Germany), linked to a time correlated single photon counting (TCSPC) module (SPC830, Becker and Hickl, Germany). Photon counts of at least 1000 used for the multi-exponential analysis. Raw time correlated single photon counting decay curve at each pixel (256×256 or higher) of the images were analysed using SPCImage software v.6.9 (Becker and Hickl, GmbH); an incomplete single exponential fit model with a laser repetition time value of 12.5 ns was used for the decay curve fitting. Lifetime values with χ2 between 0.8 and 1.3 were taken as a good exponential decay fit. Lifetimes were calculated from an average of a minimum of fifteen distinct fields of view, with ≥5 taken from three separately prepared slide samples.

Transmission Electron Microscopy (TEM) analysis of cells and isolated vesicles: Negative stained TEM samples of cells and vesicles were prepared in one of two ways. 10 μl of E. coli cells expressing VNp-mNeongreen from an overnight culture was placed onto a formvar/carbon coated 400 mesh gold grid and incubated in a humid chamber at 37° C. to allow vesicle formation. Recombinant vesicles isolated from a culture of E. coli expressing VNp-mNeongreen were placed onto a formvar/carbon coated 400 mesh gold grid and incubated in a humid chamber at 37° C. to allow vesicle formation. Recombinant vesicles isolated from a culture of E. coli expressing VNp-mNeongreen were placed onto a formvar/carbon coated 600 mesh copper grid and left for 5 mins at room temperature to allow vesicles to settle onto the surface. Both samples were then fixed in 2.5% glutaraldehyde in 100 mM sodium cacodylate buffer pH7.2 (CAB) for 10 minutes. Grids were then washed in 100 mM CAB and milliQ® water. Grids were then dried and negative stained for 5 seconds in 2% aqueous uranyl acetate.

TEM thin section analysis of E. coli cells: E. coli expressing VNp-mNeongreen were cultured as described above and harvested by centrifugation at 3,000 g for 10 min. The cell pellet (approximately 100 μl) was resuspended in 2 ml of 2.5% (w/v) glutaraldehyde in CAB and fixed for 2 hr at room temperature with gentle rotating (20 rpm). Cells were pelleted by centrifugation at 6,000 g for 2 min and were washed twice for 10 min with 100 mM CAB. Cells were postfixed with 1% (w/v) osmium tetroxide in 100 mM CAB for 2 hr and subsequently washed twice with ddH2O. Cells were dehydrated by incubation in an ethanol gradient, 50% EtOH for 10 min, 70% EtOH overnight, and 90% EtOH for 10 min followed by three 10-min washes in 100% dry EtOH. Cells were then washed twice with propylene oxide for 15 min. Cell pellets were embedded by re-suspension in 1 ml of a 1:1 mix of propylene oxide and Agar LV Resin and incubated for 30 min with rotation. Cell pellets were infiltrated twice in 100% Agar LV resin (2×2 h). The cell pellet was resuspended in fresh resin and transferred to a 1-mL BEEM embedding capsule, centrifuged for 5 min at 1100 rpm in a swing out rotor to concentrate the cells in the tip of the capsule and samples were polymerised for 20 hr at 60° C.

Ultrathin sections were cut using a Leica EM UC7 ultramicrotome equipped with a diamond knife (DiATOME) 45°. Sections (70 nm) were collected on uncoated 400-mesh copper grids. Grids were stained by incubation in 4.5% (w/v) uranyl acetate in 1% (v/v) acetic acid for 45 min followed by washing in a stream of ddH₂O. Grids were then stained with Reynolds lead citrate for 7 min followed by washing in a stream of ddH₂O. Electron microscopy was performed using a JEOL-1230 transmission electron microscope operated at an accelerating voltage of 80 kV equipped with a Gatan One View digital camera.

Immuno-EM of isolated vesicles: 2 μl of filtered media containing recombinant vesicles from a culture of E. coli expressing VNp-mNeongreen was placed onto a formvar/carbon coated 600 mesh copper grid and left for 5 mins at room temperature to allow vesicles to settle. Vesicles were osmotically shocked to rupture vesicles by moving grids into 2×20 μl drops of milliQ water for 10 minutes at room temperature. Samples were then fixed in 2% formaldehyde and 0.5% glutaraldehyde in CAB for 15 minutes at room temperature. Grids were then washed in 6×20 μl drops of CAB and 6×20 μl drops of TBST (20 mM Tris-HCl, 500 mM NaCl, 0.05% Tween 20 and 0.1% BSA pH7.4). Samples were blocked in a 20 μl drop of 2% BSA in TBST at room temperature for 30 min. Grids were then transferred directly into a 20 μl drop of anti-mNeongreen rabbit polyclonal (Cell Signalling Technology) primary antibody diluted 1:100 in TBST and incubated for 1 hr. Grids were washed in 6×20 μl drops of TBST. Grids were then moved into a drop of goat anti-rabbit IgG 5 nm gold (British Biocell International) diluted 1:50 and then moved to a fresh drop of the same antibody and incubated for 30 min. Excess antibody was removed by washing in 6×20 μl drops of TBST and 6×20 μl drops of milliQ water and dried.

Grids were negative stained for 5 seconds in 2% aqueous uranyl acetate. Electron microscopy was performed using a JEOL-1230 transmission electron microscope operated at an accelerating voltage of 80 kV equipped with a Gatan One View digital camera.

Immuno-Electron Microscopy (EM) of E. coli cells: E. coli expressing VNp-mNeongreen were cultured as described above and harvested by centrifugation at 3,000 g for 10 min. The cell pellet (approximately 100 μl) was resuspended in 2 ml 2% (w/v) formaldehyde and 0.5% glutaraldehyde in CAB and fixed for 2h at room temperature. The sample was washed 2×10 minutes in CAB. Cells were dehydrated by incubation in an ethanol gradient, 50% EtOH for 10 min, 70% EtOH overnight, and 90% EtOH for 10 min followed by three 10-min washes in 100% dry EtOH. Cells were then suspended in LR White resin medium grade (London Resin Company) for 4 h and then in fresh LR White resin overnight. Following 2×4 h changes in fresh LR White resin samples were placed in sealed gelatine capsules and spun in a swing out rotor at 1100 rpm to concentrate cells. Gelatine capsules containing the cell pellets were polymerised upright at 60° C. for 20 hours. Ultrathin sections were cut using a Leica EM UC7 ultramicrotome equipped with a diamond knife (DiATOME) 45°). Sections (80 nm) were collected on uncoated 400-mesh gold grids.

Samples were blocked in a 20 μl drop of 2% BSA in TBST at room temperature for 30 min. Grids were then transferred directly into a 20 μl drop of anti-mNeongreen rabbit polyclonal (Cell Signalling Technology) primary antibody diluted 1:10 in TBST and incubated for 1 hr. Grids were washed in 6× TBST. Grids were then moved into a drop of goat anti-rabbit IgG 5 nm gold (British Biocell International) diluted 1:50 and then moved to a fresh drop of the same antibody and incubated for 30 min. Excess antibody was removed by washing in 6×20 μl drops of TBST and 6×20 μl drops of milliQ water and dried.

Grids were stained for 15 min in 4.5% uranyl acetate in 1% acetic acid solution and then washed in 6×20 μl drops of milliQ water. Grids were then stained with Reynolds lead citrate for 3 min and washed in 6×20 μl drops of milliQ water. Electron microscopy was performed using a JEOL-1230 transmission electron microscope operated at an accelerating voltage of 80 kV equipped with a Gatan One View digital camera.

Dynamic Light Scattering studies: All studies were carried out using Anton Paar Litesizer™ 500 and processed using Kalliope™ Professional. All vials/cuvettes/microfuge tubes used for sample preparation were clean and dry. All solvent systems used were filtered to remove any particulates that may interfere with the results obtained.

VNp-fusion export with fermentation culture: 160 ml of an overnight 450 ml LB (supplemented with 1% w/v glucose and 15 mg/L tetracycline) preculture of E. coli containing pAVE011-VNpDARPinOFF7 grown in a 2 L baffled flask overnight culture in a shaking incubator at 37° C., 200 rpm (2.5 cm throw) was used to inoculate a 16 L Terrific Broth basal medium (without glucose or glycerol addition but with tetracycline addition) fermenter culture.

Fermenter growth conditions: pH set to 7.0 (controlled by 25% ammonia solution or 8.5% w/v orthophosphoric acid solution); pO2 set point 30% (cascaded to agitator then via oxygen supplementation of inlet gas); Aeration rate: 1.0 v/v/m; Agitation rate: 200-630 rpm;

Expression Feed rate of glucose/yeast extract: capped at 6 g/L/h. Feed medium consisted of 400 g/L glucose and 200 g/L yeast extract was fed to batch culture at 96 g/h. VNp2-DARP has a predicted size of 22.5 kDa.

Expression of VNp-DARP was induced by addition of IPTG (20 μg/mL final concentration) during batch phase as indicated by first spike of Dissolved Oxygen Tension % saturation at 5.3 h (elapsed fermentation time). Following depletion at 6.6 h, the linear feed was started. Fermentation duration: 18.95 h from IPTG induction (17.65 h from start of linear feed rate).

Cell pellets were harvested by 3,000 RCF centrifugation for 20 min at 10° C. An aliquot of SN harvest material was passed through a 0.45 μm PES filter for analysis.

### Results

The present invention resides in the finding that recombinant expression of part or all of the amino terminal of human synucleins brings about the continuous formation and release of extracellular vesicles from E. coli cells into the culture media (FIG. 1a). Alpha synuclein was investigated in particular, in which the first 60 residues of the sequence represents the entire alpha-helical membrane interacting domain and the first 38 residues is the first half of this domain, truncated at a predicted loop region. Both these sequences promoted export of mNeongreen vesicles to the same extent.

The sequence having the first 38 residues (VNp2) interacted with the cell membrane (FIGS. 10 and 11) and, from 4 hours, VNp expression (cytosolic VNp2concentration reached 18.8±0.14 μM) induced localised curvature of the bacterial membrane that extended outwards until formation of a distinct vesicle, which was released into the growth media upon membrane scission (FIGS. 1 b-d). This process occurred repeatedly without impacting cell viability, allowing large scale production of vesicles from the same cell (FIG. 1d and FIG. 12).

Fusion of sequences encoding VNp to those encoding the monomeric fluorescent protein mNeongreen (Shaner, N. C. et al (2013) Nat Meth 10, 407-409) led to the production and export of large VNp-mNeongreen containing fluorescent vesicles into the culture media (FIGS. 1e, 1f). Immuno-EM analysis of confirmed the mNeongreen cargo was located exclusively within the lumen of the vesicles (FIGS. 1e-1f). Immuno-electron microscopy confirmed the exclusive localisation of the mNeongreen cargo within the lumen of the vesicles (FIG. 1f, FIG. 13) to support the interpretation that the VNp interacts with lipids in the inner membrane to accumulate to a critical concentration that induces localised outward curvature of the membrane and subsequent vesicle formation. The VNp was then released from the inward curving membrane into the lumen of the forming vesicle, which is, in turn, released into the media upon scission and reclosure of the cell membrane (FIG. 1j).

While centrifugation separated vesicles from the majority of bacterial cells, rapid single step filtration with sterile 0.45 μm polyethersulfone (PES) filters was used to isolate efficiently and effectively the vesicles from bacteria (FIG. 1 g-h, FIG. 11). Remarkably, these isolated vesicles had uniform size and provided a stable environment for effective long term protein storage of functional folded recombinant protein (FIG. 11). The proteomic and densitometric analysis of the contents of the recombinant vesicles shows the relative purity of the recombinant protein, which is ˜40-60% of total number from proteomics and gel analysis (see FIG. 2). The degree of purity of the fusion protein harvested by one step filtration is highly likely to be sufficient for a very wide range of applications (FIG. 1i) or supports subsequent purification after sonication of the vesicles when necessary.

This system provides a simple and attractive mechanism for releasing membrane packaged recombinant proteins into the media for enhancing recombinant protein production and subsequent processing. The mNeongreen provided a rapid quantification of soluble target protein exported into the media with different physical properties and expression challenges (such as membrane binding, disulphide-bond containing, or otherwise insoluble or toxic proteins) to test the applicability of this technology for the expression of the spectrum of molecules demanded by the life sciences community. Expression of each protein was tested as VNp, or VNp-mNeongreen amino terminal fusions and compared to the expression of equivalent non-VNp fusion proteins (FIG. 1k-m, Table 2).

Experiments have also shown that the technology may be applied to a wide range of E. coli strains (strains (e.g. BL21, λ, JM109 and K12 lineages), using expression from different inducible and constitutive promoters. The VNp system functions in W3110 cells, which allows generation of recombinant protein filled vesicles with a reduced immunogenic response. VNp-fusions can be expressed from a variety of plasmids (including pUC19 and pBR322 based derivatives) and modulated VNp-fusion expression can be driven from diverse promoters (e.g. T7, rhamnose) and induction levels (see FIG. 3 and Table 1).

TABLE 1
Yields of exported protein from W3110 lambda DE3 E. coli cells:

| VNp-fusion | Yields (mg protein/L culture) |
|---|---|
| VNp-DARP | 262.8 + 35.1 |
| VNp-mNeongreen | 128.2 |
| VNp6-mNeongreen | 478.8 |
| VNp15-mNeongreen | 445.4 |

The inventors have also demonstrated that the protein of interest remains soluble (i.e. VNp is not necessary for maintaining protein solubility) on removal of the VNp tag using protease digest once the protein of interest has been released from the vesicles. As demonstrated in FIG. 7, purified VNpmNG-TEV-DARP and VNp-mNG-TEV-Uricase were digested with TEV protease. The resultant cleaved proteins were not detectable within the pellet fraction (P) and remained in the supernatant (S/N) fraction after centrifugation at 13,200 RCF.

While a fluorescent protein allows rapid quantification of soluble target protein exported into the media, a wider range of targeted proteins were selected with different physical properties and expression challenges and used to validate this technology. Test target proteins included Designed Ankyrin Repeat Protein off7 (DARP), Stefin A, Uricase (FIG. 1k), Erythropoietin (EPO), Etanercept, Fibroblast Growth Factor 21 (FGF21), and human growth hormone (hGH). As such, these proteins are representative of different physical properties and expression challenges (such as membrane binding, dimeric, disulphide-bind containing, or otherwise insoluble proteins), which are not normally expressed to meaningfully detectable levels in E. coli and therefore demonstrate the wider applicability of this technology. Expression of each protein was tested as VNp, or VNpmNeongreen amino terminal fusion proteins and compared to the expression of equivalent non-VNp fusion proteins (Table 2).

TABLE 2
Summary of soluble protein yields.

| | | Vesicle | Total |
|---|---|---|---|
| Protein | Cytosolic | exported (%) | yield |
| mNeongreen (mNG) | 59.19 ± 9.55 | n.d. (0%) | 59.2 |
| DARP | 31.66 ± 10.81 | n.d. (0%) | 31.7 |
| Uricase | 402.14 ± 100.68 | n.d. (0%) | 402.1 |
| StefinA | 373.81 ± 14.75 | n.d. (0%) | 373.8 |
| EPO | n.d. | n.d. (0%) | 0 |
| FGF21 | n.d. | n.d. (0%) | 0 |
| Etanercept | n.d. | n.d. (0%) | 0 |
| hGH | n.d. | n.d. (0%) | 0 |
| VNp2-mNG | 55.09 ± 4.26 | 416.72 ± 0.01 (88%) | 471.8 |
| VNp2-DARP | 75.97 ± 43.82 | 865.26 ± 11.72 (92%) | 941.2 |
| VNp2-Uricase | 577.18 ± 146 | 322.78 ± 122.66 | 900.0 |
| | | (36%) | |
| VNp2-StefinA | 98.73 ± 6.38 | 406.33 ± 122.66 | 505.1 |
| | | (80%) | |
| VNp2-FGF21 | n.d. | 51.74 ± 5.71 (100%) | 51.7 |
| VNp2-EPO | n.d. | n.d. (0%) | 0 |
| VNp2-Etanercept | n.d. | n.d. (0%) | 0 |
| (10 μg/ml IPTG) | | | |
| VNp2-hGH | n.d. | n.d. (0%) | 0 |
| (10 g/ml IPTG) | | | |
| mNG-DARP | 127.58 ± 21.30 | n.d. (0%) | 127.6 |
| mNG-Uricase | 32.70 ± 1.81 | n.d. (0%) | 32.7 |
| mNG-StefinA | 11.81 ± 0.99 | n.d. (0%) | 11.8 |
| mNG-EPO | 15.03 ± 1.78 | n.d. (0%) | 15.0 |
| mNG-FGF21 | 8.67 ± 0.88 | n.d. (0%) | 8.7 |
| mNG-Etanercept | 13.61 ± 2.82 | n.d. (0%) | 13.6 |
| (10 μg/ml IPTG) | | | |
| mNG-hGH | 16.395 ± 3.05 | n.d. (0%) | 16.4 |
| (10 μg/ml IPTG) | | | |
| VNp2-mNG-DARP | 164.4 ± 44.96 | 537.08 ± 28.26 (77%) | 701.5 |
| VNp2-mNG-Uricase | 102.88 ± 16.46 | 109.24 ± 29.24 (52%) | 212.1 |
| VNp2-mNG-StefinA | 70.25 ± 24.26 | 101.97 ± 31.78 (59%) | 172.2 |
| VNp2-mNG-EPO | 12.54 ± 2.30 | 19.43 ± 5.84 (61%) | 31.9 |
| VNp2-mNG-FGF21 | 10.0 ± 0.90 | 13.11 ± 4.99 (57%) | 23.1 |
| VNp2-mNG- | 170.27 ± 22.31 | 7.13 ± 4.820 (4%) | 177.4 |
| Etanercept | | | |
| (10 μg/ml IPTG) | | | |
| VNp2-mNG-hGH | 57.69 ± 8.48 | 2.57 ± 0.92 (4%) | 60.3 |
| (10 μg/ml IPTG) | | | |
| VNp2-mNG | 352.18 ± 27.97 | 112.8 ± 19.08 (24%) | 464.91 |
| (Nt acetylated) | | | |
| VNp2-LZ-mNG | 56.04 ± 28.5 | 337.08 ± 17.3 (86%) | 393.12 |
| VNp2-LZ-mNG- | 20.61 ± 6.60 | 36.22 ± 5.86 (55%) | 56.8 |
| Etanercept + | | | |
| VNp2-LZ | | | |
| VNp2-LZ-hGH | n.d. | 10.14 ± 1.59 (100%) | 10.1 |
| VNp2-LZ-mNG-hGH + | 17.78 ± 6.96 | 15.96 ± 3.21 (47%) | 33.7 |
| VNp-LZ | | | |
| VNp2-mNG | 169.94 ± 113.72 | 240.81 ± 23.85 (59%) | 410.74 |
| (50 μg/ml IPTG) | | | |
| VNp2-mNG | 59.43 ± 34.51 | 227.30 ± 27.57 (79%) | 286.73 |
| (100 μg/ml IPTG) | | | |
| VNp4-mNG | 283.6 | 106.3 (27%) | 389.9 |
| VNp5-mNG | 252.2 ± 159 | 429.4 ± 155 (63%) | 681.5 |
| VNp6-mNG | 52.9 ± 21.7 | 585.8 ± 15.1 (92%) | 638.8 |
| VNp7-mNG | 123.8 ± 8.6 | 362.5 ± 26.1 (75%) | 486.5 |
| VNp8-mNG | 185.7 ± 85.1 | 344.0 ± 30.4 (65%) | 529.8 |
| VNp9-mNG | 89.2 ± 30.4 | 652.1 ± 27.3 (88%) | 741.2 |
| VNp10-mNG | 52.7 ± 7.8 | 728.6 ± 7.8 (93%) | 781.4 |
| VNp11-mNG | 83.5 ± 28.8 | 483.4 ± 148.2 (85%) | 566.8 |
| VNp12-mNG | 60.7 ± 19.9 | 458.0 ± 21.4 (88%) | 517.7 |
| VNp13-mNG | 55.1 ± 19.8 | 426.7 ± 45.6 (89%) | 481.8 |
| VNp14-mNG | 75.8 ± 20.3 | 621.2 ± 38.0 (89%) | 697.0 |
| VNp15-mNG | 75.2 ± 10.9 | 543.2 ± 20.4(88%) | 618.3 |
| VNp16-mNG | 73.9 ± 3.2 | 556.4 ± 31.3(88%) | 630.3 |
| VNp2-mNG (30° C.) | 313.7 ± 46.3 | 176.1 ± 28.2 (36%) | 489.7 |
| VNp5-mNG (30° C.) | 265.5 ± 27.7 | 293.3 ± 23.3 (52%) | 558.9 |
| VNp6-mNG (30° C.) | 111.3 ± 24.9 | 443.2 ± 13.1 (80%) | 554.4 |
| VNp2-mNG (25° C.) | 241.3 ± 0.0 | 117.0 ± 8.6 (33%) | 358.3 |
| VNp5-mNG (25° C.) | 131.2 ± 33.8 | 9.0 ± 3.0 (6%) | 140.3 |
| VNp6-mNG (25° C.) | 333.5 ± 9.8 | 186.9 ± 2.0 (36%) | 520.4 |

Average yields ± s.d. calculated from ≥3 independent biological repeats. Yields measured as mg of soluble target protein/litre. Bacterial shaking flask culture at 37° C. with T7 promoter induced with 20 μg/ml IPTG unless stated otherwise.
n.d.: not detectable;
LZ = leucine zipper sequence;
mNG = mNeongreen

The relative export yield of mNeongreen fusions with an extended range of Vesicle Nucleating peptide sequences was determined. As can be seen from Table 3, there was a drop off in export yield at shorter VNp lengths. While some export was observed with 9 amino acid peptide lengths, at this shorter length vesiculation activity was reduced significantly.

TABLE 3

| | Average cytosolic yield (mg/L) | Average export yield (mg/L) |
|---|---|---|
| VNp 2 | 47.3 ± 12.0 | 418.9 ± 37.0 |
| VNp 4 | 283.6 | 106.3 |
| VNp 5 | 353.7 ± 17.5 | 264.8 ± 82.2 |
| VNp 6 | 52.9 ± 21.7 | 585.8 ± 15.1 |
| VNp 7 | 123.8 ± 8.6 | 362.6 ± 26.1 |
| VNp 8 | 185.7 ± 85.1 | 344 ± 30.4 |
| VNp 9 | 89.2 ± 30.4 | 652.1 ± 27.3 |
| VNp 10 | 128.1 ± 106.8 | 586.3 ± 199.8 |
| VNp 11 | 83.5 ± 28.8 | 483.4 ± 148.2 |
| VNp 12 | 60.7 ± 19.9 | 458 ± 21.4 |
| VNp 13 | 55.1 ± 19.8 | 426.7 ± 45.6 |
| VNp 14 | 75.8 ± 20.3 | 621.2 ± 38.0 |
| VNp 16 | 75.2 ±10.9 | 543.2 ± 20.4 |
| VNp 6 | 73.9 ± 3.2 | 556.4 ± 31.3 |
| VNp 17 | 252.5 | 482.6 ± 28.2 |
| VNp 18 | 76.9 | 573.6 ± 77.5 |
| VNp 19 | 196.3 | 400.4 ± 25.3 |
| VNp 20 | 130.5 ± 16.8 | 474.6 ± 122.7 |
| VNp 21 | 228.2 ± 56.4 | 252.1 ± 2.0 |
| VNp 22 | 205.7 ± 126.7 | 300.1 ± 41.7 |
| VNp 23 | 344.2 | 318 |
| VNp 24 | 213.8 ± 55.0 | 382.3 ± 49.4 |
| VNp 25 | 305.7 ± 22.9 | 149.4 ± 59.8 |
| VNp 26 | 139.7 ± 97.6 | 440.5 ± 303.0 |
| VNp 27 | 377.5 ± 20.0 | 129.8 ± 46.0 |
| VNp 28 | 299.6 | 46 |

Fusion with VNp enhanced protein expression and secretion for each target protein highly effectively. Thus, VNp protein expression supports the expression of individual proteins ranging from less than 1 kDa (VNp-His6) to 85 kDa (VNp-mNeongreen-Etanercept) in size, as well as protein complexes as demonstrated by fluorescence from pairs of Bimolecular Fluorescence Complementation VNp-fusions within exported vesicles (FIG. 11) (Kodama, Y. & Hu, C.-D. (2010) BioTechniques 49, 793-805). Importantly, VNp fusion enhanced the overall yield of soluble forms of each target protein examined, with yields of almost 1 g soluble protein/litre of shaking flask culture obtained in the case for DARP (Table 2). The versatility of the system was demonstrated by the production of membrane binding proteins (FGF21) and VNp-uricase with full enzymatic activity. Furthermore, VNp-Etanercept exhibited appropriate ligand binding properties (FIG. 13) and mNeongreen fluorescence shows how functional protein folding accompanied VNp induced vesicle packaging and was maintained in the isolated vesicles.

The VNp fusion allows production of soluble folded proteins that are otherwise insoluble or reduce the viability of bacterial cells (e.g. DNase, Etanercept, EPO and hGH). In the case of the disulphide bond containing proteins Etanercept and hGH (Goffe, B. & Cather, J. C. (2003) J Am Acad Dermatol 49, S105-S111; Ultsch, M. H. & Somers, W. J. (1994) Mol. Biol. 236, 286-299), the majority of the soluble recombinant protein remained within the cell. EM data show VNp-mNG-Etanercept impacts VNp remodelling of the inner membrane to induce VNp-fusion contained internalised cytosolic membrane structures (FIG. 6a-b). Similarly amino-terminal-acetylation of the VNp by recombinant NatB complex (Eastwood. T. A. et al (2017) FEBS Letters 106, 8157-9) which, like VNp-Etanercept, brings about formation of internal membrane structures within E. coli (FIG. 6c-d), increasing the proportion of the mNeongreen within the cell (FIG. 6e, Table 2).

A VNp2 tag also permits expression and isolation of heterodimeric-soluble-functional antibodies from E. coli. Fab (Fragment antigen-binding; folded chicken egg lysozyme) and mAB (monoclonal antibody; Muc1) antibodies, made up of heavy and light chain heterodimers (linked with disulphide bonds), were found to be soluble and able to bind protein G/A which is indicative of correct structure, complex formation, and functionality (see FIG. 4).

Stable alpha-helical VNp-dimers were created by introducing a leucine-zipper sequence between VNp and cargo (FIG. 12) and, consistent with the previous dimeric VNp-fusions, induced formation of mNeongreen containing inner membrane associated vesicular structures within the bacterial cytosol (FIG. 6f-h). In particular, the addition of a Leucine Zipper sequence onto the carboxyl terminal of the VNp leads to VNp dimer formation, which predominately generate internal cytosolic vesicles. As seen in FIG. 9, while the VNp-mNG (black) elution profile was consistent with a monomeric protein, the VNp-LZ-mNG (grey) eluted from the column in earlier fractions consistent with it existing predominantly as a dimer. Accordingly, the present invention provides an attractive simple system for expression of toxic and/or disulphide-bond containing proteins from E. coli (FIG. 6i).

VNps have also been generated to allow in-vesicle targeted proteolytic cleavage of recombinant proteins. E. coli cells containing constructs to express a VNp-mNeongreen-DARPin^{off7} fusion alone or in combination with VNp-Maltose Binding Protein (MBP)-TEV protease fusion were cultured and expression induced in an overnight flask culture. The VNp-mNeongreen-DARPin^{off7} fusion contained a TEV cleavage site engineered between the mNeongreen and DARPin sequences. As seen in FIG. 5, expression of the VNp-mNG-DARP alone resulted in expression and vesicular export of full-length protein (50.8 kDa size). In contrast co-expression of the same protein together with the TEV protease fusion resulted in cleavage of the VNp-mNG-DARP at the TEV site, and export of the subsequent export of the VNp-mNG (32 kDa) and DARPin^{off7} (18.8 kDa) fragments into the exported vesicles within the culture media.

Accordingly, the invention provides an attractive method for generating recombinant protein containing internal membrane bound structures for expression and compartmentalisation of disulphide bond containing, insoluble (e.g. Etanercept, hGH and antibody complexes), or otherwise toxic (e.g. DNase) proteins. However, co-expression of VNp-LZ dimeric cargo fusions with an additional VNp-LZ peptide (to increase overall extracellular vesicle production) resulted in the re-direction of internal compartment bound proteins towards the export route, as soluble cargo packaged vesicles isolated from the media (Table 2) to facilitate specific downstream processes for these proteins. Thus, not only does the VNp system support the immediate isolation of fusion proteins from the media, but it offers alternative internal expression systems, where this would be advantageous in certain fields such as the generation of enzyme cascades for complex synthesis or other aspects of synthetic biology.

Investigations were then made to ascertain whether simple modifications to the VNp protein to modulate lipid interactions would enhance the exported protein yields. A series of VNp variants was tested and, by modifying charges and side chain length of targeted residues along the helix surface, it was found that not only could vesicular export be enhanced over a wide range of culture temperatures (e.g. VNp6), but also the size of the VNp could be reduced to 9 residues in length (e.g. VNp26) (Table 2).

Consistent with VNp impacting membrane dynamics, optimal vesicle production was observed when cells were cultured higher temperatures for VNp2 (see Table 2 and Table 4). However, variants of VNp6 did induce vesicle production with high efficiency over a wide range of culture temperatures (25° C. to 37° C.).

TABLE 4
Yield of VNp-mNeongreen fusions at 25° C., 30° C. and 37° C.

| | Total | | | % |
|---|---|---|---|---|
| Protein | yield | Cytosolic | Exported | Exported |
| VNp-mNeogreen (37° C.) | 472 | 55 ± 4 | 417 ± 0 | 88 |
| VNp6-mNeogreen (37° C.) | 639 | 53 ± 22 | 586 ± 15 | 92 |
| VNp-mNeogreen (30° C.) | 490 | 314 ± 46 | 176 ± 28 | 36 |
| VNp6-mNeogreen (30° C.) | 554 | 111 ± 25 | 443 ± 13 | 80 |
| VNp-mNeogreen (25° C.) | 358 | 241 ± 0 | 117 ± 9 | 33 |
| VNp6-mNeogreen (25° C.) | 520 | 334 ± 10 | 187 ± 2 | 36 |

The effect of oxygenation levels was also investigated. Cells were grown in the same volume of media, within flasks with increasing size (and therefore increased surface area), ±parafilm (±anaerobic), ±baffles (to increase agitation of culture). As illustrated in FIG. 16 and shown in Table 5, it was found that increasing surface area: volume ratio had a direct impact upon export of vesicle packaged proteins. Addition of Parafilm® (i.e. anaerobic conditions) had a dramatic and negative impact upon vesicle formation, while agitation had a small but significant negative effect.

TABLE 5
Extracellular vesicle formation requires cell culture oxygenation.

| Flask size | Volume | Relative SA:Vol | Absorbance (506 |
|---|---|---|---|
| 100 ml with Parafilm | 100 ml | 1 | 0 |
| 500 ml with Parafilm | 100 ml | 6.45625511 | 0.0144 |
| 500 ml | 100 ml | 6.45625511 | 0.0664 |
| 500 ml with baffles | 100 ml | 6.45625511 | 0.0562 |
| 2000 ml | 100 ml | 15.0539657 | 0.181 |
| 2000 ml with baffles | 100 ml | 15.0539657 | 0.1593 |

The inventors have also established that the VNp technology described herein may be applied to 1 L volume shaking flask culture volumes, when in 5 L flask to maintain surface area: volume ratio (see FIG. 8 and Table 6).

TABLE 6
Yields of exported VNp-mNeongreen in a range of culture
volumes and flask sizes.

| Flask size | Volume | SA: Volume | Yield (mg/L) |
|---|---|---|---|
| 500 ml | 100 ml | 78.96 | 185.5 |
| 1000 ml | 100 ml | 128.6 | 139.1 |
| 2000 ml | 100 ml | 184.1 | 505.6 |
| 500 ml | 25 ml | 303.92 | 416.0 |
| 5000 ml | 750 ml | 50.69 | 204.5 |
| 5000 ml | 1000 ml | 38.01 | 192.0 |

It will be appreciated that expression levels needed to be modulated according to the cargo being expressed to identify optimal conditions for vesicle formation. Consistent with VNp impacting membrane dynamics, optimal vesicle production was observed when cells were cultured at higher temperatures, where membranes are more dynamic. However, as expression can be supported from a variety of promoters (e.g. T7, rhamnose, arabinose and Tac) in a range of plasmids, including pUC19 or pBR322 based derivatives, specific expression levels can be easily tested with a variety of expression systems.

FIG. 15 shows that a VNp2-DARP construct successfully exported protein in a 20 L fermenter/bioreactor. Protein was seen in both the cell pellet and the supernatant, in which the highest concentration of protein was found in the supernatant.

Experiments have confirmed the stability of the recombinant vesicles, and the fusion protein within them, when stored at 4° C. (see FIG. 17). Dynamic light scattering (DLS) confirmed the presence of two species with differing intensity distribution maxima in solution. The first exhibited a hydrodynamic diameter of 166 nm, attributed to the isolated vesicles and correlating with those species present in EM images, and the second exhibited a hydrodynamic diameter of ≈10 nm, attributed to vesicle fragments. Average polydispersity indices from each sample were greater than 1, indicating vesicles with a broad distribution of sizes in the culture media. The average hydrodynamic diameter of the vesicles did not vary after 6 months storage at 4° C. in sterile phosphate buffered saline (PBS) (hydrodynamic diameter=169 nm), which was consistent with EM imaging of the vesicles. In addition, there was no significant difference in the zeta potential (calculated from peak maxima) of day-old vesicles (−10.5 mV), compared with 4-month old vesicles (−11.1 mV), and no observable significant loss in vesicle contained VNp-mNeongreen over a 3-month period. Therefore, the isolated vesicles provide a stable environment for effective long term protein storage of soluble recombinant protein. Vesicle isolated VNp-uricase was not only as enzymatically active as uricase purified from a cell pellet, but this activity was maintained to a higher degree by VNp-uricase stored within isolated vesicles for 2 months at 4° C. when compared to purified protein stored at 4° C. in buffer over the same period (FIG. 17), highlighting the stable environment the vesicles afford their protein cargo.

VNp sequences can be used to induce the formation of extracellular VNp-fusion containing vesicles from Mammalian cell expression systems in culture (see FIG. 18). HEK293T cells were transiently transformed (Lipofectamine 2000 mediated transfection) with VNp2-mNeongreen and VNp6 expression constructs under the control of either CMV or SV40 promoters. Expression and relative induction levels was confirmed by western blot (FIG. 18A). VNp-mNeongreen transfected HEK cells were grown on coverslips in DMEM media containing 10% fetal Bovine serum. Cells were imaged using widefield microscopy to visualise mNeongreen expression. The arrows in FIG. 18 highlight VNp-mNeongreen containing recombinant vesicles. Equivalent results were observed in CHO-S cells when expressing VNp6-mNeongreen under the control of the SV40 promoter.

The inventors have also established that the VNp tag technology dramatically enhances recombinant protein expression of E. coli cultured in anaerobic conditions. Conditions have been identified that promote cytosolic compartmentalisation of VNp-fusions between VNp sequences and either mNeongreen or DARPin^{off}, neither of which are seen to express in anaerobic conditions in the absence of the VNp tag. FIG. 19 shows a gel of expression profiles from cells containing expression constructs for mNeongreen and StefinA alone, or fused to a VNp, and shows in each case that addition of the VNp facilitates expression of these recombinant proteins in cells cultured in anaerobic growth conditions.

### Discussion

As has been demonstrated, the simple peptide fusion described herein increases yields and simplifies downstream processing of a wide range of recombinant proteins from E. coli. Importantly, the ease with which otherwise insoluble or toxic proteins can be isolated in milligram or gram quantities suggests that this approach is an attractive starting point for the expression of any recombinant protein of interest. A highly attractive aspect of this production system is the stability of proteins and preservation of enzymatic activity when the vesicles are maintained at 4° C. As a result, this versatile system lends itself to a wide range of downstream processes and applications, including high throughput expression screens, protein storage, generation of recombinant bioreactors, environmental dispersion of biomolecules, micro-vesicle therapies, nanobody production alongside vaccine and viral therapy delivery.

An initial investigation investigated whether the impact of amino-terminal acetylation of the essential human neuronal protein a-synuclein (αSyn) (Maltsev, A. S. et al (2012) Biochemistry 51, 5004-5013) could be observed in E. coli. Not only was it discovered that differences in the oligomerisation status could be followed within the bacterial cell (Eastwood, T. A. et al (2017) FEBS Letters 106, 8157-9), but it also resulted in the formation of large numbers of large extracellular vesicles, which form from the E. coli membrane (unpublished observations). Fluorescence imaging revealed that, once membrane associated αSyn reaches a critical level, it induces outward curvature of the bacterial membrane, leading to the formation of extracellular vesicles, (significantly larger than natural outer membrane vesicles) containing the αSyn-fluorescent protein fusion.

VNp-induced vesicle formation requires a critical concentration of the VNp-fusion protein to promote vesiculation. The Vesicle Nucleating peptides interact with the cell membrane and, from 4 hours, VNp expression (cytosolic VNp concentration typically 18.8±0.14 μM) induced localised curvature of the bacterial membrane that extended outwards until formation of a distinct vesicle, which was released into the growth media upon membrane scission. Expression of some target proteins of interest can either be toxic to the cell, interact with the cell membrane and/or fail to fold correctly at even moderate cytosolic concentrations. Accordingly, a dimerising leucine zipper (LZ) peptide was introduced between the VNp and protein sequence of interest. Results showed that when this construct was either expressed alone, or together with an additional VNp-LZ peptide, packaging and export of otherwise difficult to express proteins was enhanced (examples include Etanercept and hGH).

In addition, in vivo amino terminal acetylation within the bacterial cell (see Eastwood, T. A. et al (2017) FEBS Letters 106, 8157-9; Johnson, M. et al (2010) PLoS ONE 5, e15801) of the VNp promoted the formation of internalised VNp-fusion vesicle structures within the bacterial cytosol, and improved expression of the protein of interest.

These initial observations led to the establishment that expression of fusions between αSyn and human Growth Hormone (hGH), within E. coli cells, led to the release of αSyn-hGH containing vesicles into the culture media, in both shaking flasks and commercial bioreactor cell-culture systems. This demonstrated that vesicles containing target recombinant protein are exported into the growth media to facilitate downstream purification and processing of the protein of interest.

An investigation was undertaken to ascertain whether a specific region of αSyn brought about the vesicle formation, rather than the whole 15 kDa (140 amino acid) αSyn protein. It was found that a short 38 amino acid amphipathic alpha helical polypeptide from the amino terminus of αSyn was sufficient to interact with the cell membrane to promote formation of vesicular structures. It has subsequently been found that this 38-amino acid Vesicle Nucleating polypeptide (VNp) interacts the with inner E. coli cell membrane to impact membrane shape. Spectroscopy, EM, DLS, mass spectroscopy and live cell imaging data confirmed that expression of VNp-Fluorescent Protein (FP) fusions induced export of VNp-FP containing vesicles into the culture media. None of these phenomena were observed when FPs lacking the VNp amino-terminal fusion were expressed alone.

It was then found that equivalent polypeptides from other synuclein proteins could be used to generate fluorescent VNp-FP containing vesicles to equivalent levels. In an ongoing systematic in vitro molecular evolution approach (using the first 38 amino acids of γ-synuclein as a starting point), a growing number of modified VNp sequences were identified that significantly enhance (and those that reduce) export of vesicle packaged VNp-fusion from the E. coli cell when compared to the natural peptide sequences. Table 7 summarises the variants and the effects of each sequence on export of mNeongreen (mNG):

TABLE 7

| SEQ ID | Peptide variants | Effects on Yield and Export of mNG |
|---|---|---|
| 1 | Full length | GenBank sequence: AAL15443.1 |
| | endogenous | |
| | Hs alpha-synuclein | |
| 2 | Amino-terminal | Equivalent export and |
| | 38 aa of | yield to SEQ ID |
| | Hs alpha-synuclein | NO: 1 |
| 3 | Amino-terminal | Equivalent export and |
| | 38 aa of | yield to SEQ ID |
| | Hs beta-synuclein | NOs: 1 and 2 |
| 4 | Amino-terminal | 83% yield and 25% |
| | 38 aa of Hs | export compared |
| | gamma-synuclein | to SEQ ID NOs: 1 and 2 |
| 5 | SEQ ID NO: 2 with | 144% yield and 103% export |
| | 3K-E mutations | compared to SEQ ID NOs: 1 and 2 |
| 6 | SEQ ID NO: 4 with | 135% yield and 141% export |
| | 2K-D mutations | compared to SEQ ID NOs: 1 and 2 |
| 7 | SEQ ID NO: 4 with | 103% yield and 87% export |
| | 6K-A mutations | compared to SEQ ID NOs: 1 and 2 |
| 8 | SEQ ID NO: 4 with | 112% yield and 82% export |
| | A29M mutation | compared to SEQ ID NOs: 1 and 2 |
| 9 | SEQ ID NO: 6 with | 110% yield and 110% export |
| | D2G mutation | compared to SEQ ID NOs: 1 and 2 |
| 10 | SEQ ID NO: 6 with | 102% yield and 102% export |
| | D2P mutation | compared to SEQ ID NOs: 1 and 2 |
| 11 | First 20 residues of | 148% yield and 149% export |
| | SEQ ID NO: 6 | compared to SEQ ID NOs: 1 and 2 |
| 12 | First 25 residues of | 131% yield and 130% export |
| | SEQ ID NO: 6 | compared to SEQ ID NOs: 1 and 2 |
| 13 | First 30 residues of | 133% yield and 133% export |
| | SEQ ID NO: 6 | compared to SEQ ID NOs: 1 and 2 |
| 14 | SEQ ID NO: | 157% yield and 156% export |
| | 4-110A | compared to SEQ ID NOs: 1 and 2 |
| 15 | SEQ ID NO: | 165% yield and 175% export |
| | 4-A11PQ24P | compared to SEQ ID NOs: 1 and 2 |
| 16 | SEQ ID NO: | 120% yield and 116% export |
| | 4-E31K | compared to SEQ ID NOs: 1 and 2 |

It can be seen that the shorter versions of VNp6 work particularly well, with 95% of the recombinant protein being exported into the media. The α-helical structure of the VNp is enhanced by interaction with specific lipids and the VNp interacts with inner bacterial membrane enabling expression of a protein of interest and export from the host cell into culture media.

As well as optimising the VNp sequence itself, a range of E. coli strains and culture conditions have been tested and compared to identify optimal conditions for the production of membrane packaged target proteins. Standard E. coli cultures media, such as Luria Broth (LB) all seem to work well, especially richer media such as Terrific Broth (TB): about four times more VNp-fusion protein was released when cultured in TB compared to LB. Not only was more overall recombinant protein produced in the media, but a larger proportion of it was released into the media in TB. In contrast, while the VNp induced vesicle packages were obtained when cells were cultured in minimal/complex medias, it is to a significantly lower level.

Some specific media components, such as citric acid, were found to affect, reduce or inhibit release, whereas others, such as phosphate ion concentration, did not impact vesicle release. In some embodiments, one or more, and preferably all, of the following have been found to be advantageous:
A final K₂SO₄ concentration of concentration of less than 4 g/l;
A final MgSO₄ concentration of less than 10 mM;
A final CaCl₂ concentration of less than 0.03 g/l;
A NaCl concentration of up to 5 g/l;
A glycerol concentration of less than 0.04% (v/v); and
A final sorbitol concentration of up to 1 mM (preferably up to 0.1 mM).

Cell culture temperature can also affect the efficiency of specific VNp variant dependent export. For example, SEQ ID NO:2 is temperature dependent, releasing better at 37° C., while, in some embodiments, improved export of vesicle-packaged proteins occurs when cells are cultured at temperatures ranging from 25° C. to 37° C. for VNp6 and VNp15 variants.

It was also found that higher oxygenation levels, to allow maximum respiration/metabolism, had a significant positive impact upon vesicle packaged protein release into the culture media.

The experiments showed that expression levels for the VNp fusion proteins were important for ensuring optimal vesicle release. For the core VNp constructs, cell viability, target protein expression and export at various induction levels were studied and compared. The best yields were obtained by inducing expression at a level which had a just detectable impact upon cell growth, which balanced vesicle production against overall stress to the bacterial plasma membrane and viability caused by the VNp. Not only was total yield of VNp-fusions reduced significantly at higher induction rates, but the proportion of target protein produced that was exported into the media also dropped. Optimal induction for the majority of proteins was identified as 20 μg/ml IPTG for the VNp SEQ ID NO:2 fusions. However optimal yields for some toxic target proteins were achieved with 10-15 μg/ml IPTG (e.g. Etanercept, hGH).

The expression of the VNp fusions was regulated by modulating the promoter and/or inducer (e.g. T7 promoter expression regulated using IPTG ranging from 10-100 μg/ml; rhamnose promoter expression regulated by addition of 0.001% to 0.2% (Final W/V) rhamnose to the culture media.) Unsurprisingly, too high a level expression leads to excess membrane disruption (as determined from growth curves, microscopic identification of dead cells, and/or appearance of additional proteins released into the culture media from lysed dead cells—observed by SDS-PAGE analysis) and, while that leads to a transient high level of vesicle production, the impact this has upon the cell membranes lead to cells bursting and the onset of decline phase of the culture. In contrast, too low expression of the VNp fusion (i.e. ≤2 μg/ml IPTG) did not allow sufficient VNp to interact together with the membrane, and therefore vesicles did not form. As a starting point with a new VNp-fusion, expression levels that were approximately 5 to 10 times lower than the maximum allowed by IPTG induction of the T7 promoter were used.

Different strategies and reagents for targeting proteins to vesicles have been assessed. This included comparison of methods to deliver target protein into the VNp induced vesicles included direct fusions with the VNp (as described for VNp-FP fusions above); co-expression of the VNp with cytoplasmic recombinant proteins of interest; co-expression of the VNp with membrane targeted recombinant proteins; as well as optogenetics approaches (to induce

VNp-target protein interactions in response to light of specific wavelengths):
Overexpression of target protein (i.e. cytosolic localisation) resulted in a proportion incorporating into vesicles. However, this was nowhere near efficient as direct fusion. Also, there was no positive effect from the VNp on facilitating growth with hard-to-express or toxic target proteins.
Fusions were made between the target protein and bacterial membrane proteins (OmpA, CydAB, MinD). However, this method was this not as efficient as direct fusion, the membrane proteins were large, and overexpression of the membrane protein-target protein fusions can disrupt cell viability.
Optogenetics worked more efficiently than either of the above. However, optogenetic tags are large and there was a significant reduction in relative yields of the target protein.

None of the above were as efficient as direct fusion between the VNp and protein of interest, that not only targeted protein to vesicle, but enhanced folding, and protein yields, and allowed expression of toxic proteins, as they were removed from the bacterial cytosol.

A proteolytic cleavage (TEV) site was also introduced between VNp and cargo molecule to allow purification of untagged, unlabelled protein of interest from vesicles. Experiments have shown that the Etanercept remains soluble after removal of the VNp tag.

Using the short VNp sequences and under these optimised conditions, not only was a significant proportion of the fluorescent protein cargo exported into the media, but also the overall yield of the target protein within the culture (i.e. in both cells and released into media) was significantly improved when compared to equivalent proteins lacking the amino-terminal VNp fusion (i.e. in yield in cells alone) (Table 2). All VNp sequences tested promoted export, with the proportion of protein exported being greater for SEQ ID NO:10.

In optimal growth conditions (TB media, 37° C., good oxygenation, 10-20 (typically 20) μg/ml IPTG (depending upon cargo), cargo-containing vesicles were released from the bacterial cell within 10 minutes from the start of their formation. There is therefore the potential to release a large amount of cargo containing vesicles from a continuous culture in a relatively short period of time. Centrifugation purification strategies requires harvesting a significant number of cells as well media from the culture, is time consuming, and labour intensive. A simple cell filtration protocol has been developed where a fraction of the cell culture can be removed (and replaced with fresh media) and passed through a 0.45 μm filter to separate cells from vesicle containing media (see Methods above). No colony forming units were detected in 1 ml of vesicle containing media filtrates from all experiment tested. Thus, this provides a simple efficient automatable high throughput purification method.

The VNp induced vesicles and packaged proteins isolated in the media using this method were stable at 4° C. for months: the vesicle isolated VNp-uricase was not only as enzymatically active as uricase purified from the cell pellet, but this activity was maintained to a higher degree by VNp-uricase within isolated vesicles stored for 2 months at 4° C. when compared to purified protein stored in the same conditions over the same period (see FIG. 12d). This, again, highlights the stable environment the vesicles afford their protein cargo.

The VNp dependent targeted export technology of the present invention was tested using a range of proteins of bio-industrial relevance with different biophysical properties and functions. These range in size from less than 10 amino acids to almost 100 kDa size). Direct fusions were made between the VNp and protein of interest, and cells were cultured using conditions described above, with some optimisation of expression levels for different proteins. For each protein examined, the VNp fusion not only resulted in a significant proportion of the cargo being exported into the culture media, but the overall yield of soluble form for each target protein was significantly enhanced (Table 2). The functionality of some proteins was tested (e.g. fluorescence of FP, enzymatic activity of Uricase; protein A binding capacity of Etanercept) and each had expected measurable activity.

In addition to confirming that the VNp technology works with different types of promoter (e.g. CMV, rhamnose, arabinose, Tac and different strength T7 promoters) and backbone plasmid (e.g. pcDNA3.1, pUC19 or pBR322 based), the system has also been validated using a range of different E. coli strains (e.g. BL21DE3, W3110+−DE3, K12, DH10b and JM109).

Finally, scale-up of the vesicle export system has also been successfully demonstrated suggesting that the technology is suitable for industrial scale protein production.

In conclusion, the present invention provides a technology that uses a simple peptide fusion to increase yields and simplify downstream processing of a wide range of recombinant proteins from E. coli. This allows continuous protein purification during active culturing, as well as a simplified protein isolation and downstream processing (i.e. no need to disrupt cells) of a wide range of recombinant proteins from E. coli. As a result, this system provides a simple and attractive mechanism for releasing membrane packaged recombinant proteins into the media for enhancing recombinant protein production and subsequent processing.

Importantly, the ease with which otherwise insoluble or toxic proteins may be isolated in milligram or gram quantities suggests that this approach is an attractive starting point for the expression of any recombinant protein of interest. A highly attractive aspect of this production system is the stability of proteins and preservation of enzymatic activity when the vesicles are maintained at 4° C. The technology also lends itself to a wide range of downstream processes and applications, including high throughput expression screens, protein storage, generation of recombinant bioreactors, environmental dispersion of biomolecules, micro-vesicle therapies, nanobody production alongside vaccine and viral therapy delivery.

What is claimed is:

1. An isolated vesicle nucleating polypeptide (VNp) comprising an amphipathic alpha helix polypeptide derived from the amino terminus of synuclein isoforms and variants thereof.

2. The isolated vesicle nucleating polypeptide of claim 1, wherein the polypeptide has between 9 and 140 amino acids, optionally between 16 and 40 amino acids, more preferably about 20 amino acids.

3. The isolated vesicle nucleating polypeptide of claim 1, wherein the sequence is any one of SEQ ID NOs: 2 to 28.

4. The isolated vesicle nucleating polypeptide of claim 1, wherein the polypeptide is acetylated at its amino terminus.

5. The isolated vesicle nucleating polypeptide of claim 1, wherein the polypeptide further comprises a leucine zipper sequence, optionally wherein the leucine zipper sequence is at the carboxyl end of the polypeptide, and optionally wherein the leucine zipper sequence has the sequence of SEQ ID NO: 29.

6-7. (canceled)

8. The isolated vesicle nucleating polypeptide of claim 1, wherein the polypeptide further comprises a protease cleavage site.

9. A fusion protein comprising a vesicle nucleating polypeptide as claimed in claim 1 fused to the amino terminus of a biological molecule, optionally wherein the vesicle nucleating polypeptide is fused to the amino terminus of the biological molecule via a protease cleavage site.

10. (canceled)

11. The fusion protein of claim 9, wherein the protease cleavage site is for a viral protease or a viral 3C-like protease.

12. The fusion protein of claim 9, wherein the protease cleavage site is scarless.

13. The fusion protein of claim 9, wherein the protease cleavage site is specific for one or more membrane-bound or cytosolic proteases.

14. The fusion protein of claim 9, wherein the biological molecule is not constitutively expressed to detectable levels in an expression system in which the fusion protein is expressed, optionally wherein the biological molecule is a membrane binding protein, an insoluble protein, or a protein usually toxic to the host cell in which the fusion protein is expressed.

15. (canceled)

16. The fusion protein of claim 9, wherein the biological molecule has a size in the range of from less than 1 kDa to 100 kDa.

17. A nucleotide sequence or vector expressing the fusion protein of claim 9.

18. A host cell expressing the fusion protein of claim 9, or comprising the nucleotide sequence or vector of claim 17.

19. The host cell of claim 18, wherein the cell is a prokaryotic cell, a single celled eukaryotic organism or a cultured eukaryotic cell line, optionally wherein the host cell is E. coli.

20. (canceled)

21. The host cell of claim 18, wherein the host cell additionally expresses a VNp-leucine zipper peptide.

22. The host cell of claim 18, wherein the host cell further expresses one or more VNp-tagged proteases.

23. A method for promoting, enhancing or increasing formation of vesicles in a recombinant expression system, the method comprising expressing a polypeptide comprising
(i.) an isolated vesicle nucleating polypeptide (VNp) comprising an amphipathic alpha helix polypeptide derived from the amino terminus of synuclein isoforms and variants thereof,
(ii.) a fusion protein comprising the vesicle nucleating polypeptide, or p1 (iii.) a nucleotide sequence or vector expressing the fusion protein in a host cell, optionally wherein the method produces at least one soluble and/or functional form of a biological molecule from a cellular expression system, and wherein the biological molecule is not constitutively expressed in a host cell of the expression system.

24. (canceled)

25. The method of claim 23, wherein the host cell is a host cell as claimed in claim 18.

26. The method of claim 23, wherein the polypeptide, fusion protein, or nucleotide sequence or vector promotes, enhances or increases release of the vesicles from the host cell into culture media of the expression system.

27. The method of claim 23, wherein the method comprises culturing the host cell at 20° C. to 40° C., optionally under aerobic or anaerobic conditions.

28. (canceled)

29. The method of claim 23, wherein the method further comprises storing the vesicles within the host cells of the recombinant expression system, optionally wherein the vesicles are stored for up to at least four months.

30. The method of claim 23, wherein the method further comprises isolating or purifying the vesicles from the cell culture before storing, optionally wherein the vesicles are stored at about 4° C.

31-32. (Canceled)

33. The method of claim 23, the method further comprising releasing the fusion protein and optionally, further purifying the fusion protein.

34. The method of claim 23, the method further comprising releasing the biological molecule from the vesicle and optionally, further purifying the biological molecule.

35. A method for co-expressing and transporting a biological molecule in combination with an isolated vesicle nucleating polypeptide (VNp) according to claim 1, as cargo in membrane-bound vesicles.

36. The method of claim 35, wherein the biological molecule is a recombinant protein.

37. A method for the production of one or more biological molecules expressed as a recombinant protein, and optionally released from a host cell as part of a recombinant expression system, wherein the expression system comprises an isolated vesicle nucleating polypeptide comprising an amphipathic alpha helix polypeptide derived from the amino terminus of synuclein isoforms and variants thereof, a fusion protein comprising the vesicle nucleating polypeptide, a nucleotide sequence or a vector expressing the fusion protein, or a host cell comprising a nucleotide sequence or vector expressing the fusion protein.
